(19) United States
(12) Patent Application Publication
(10) Pub. No.: US 2024/0124541 A1
(43) Pub. Date: Apr. 18, 2024

(54) COMPOSITIONS AND METHODS FOR TREATING OBESITY

(71) Applicant: University of Florida Research Foundation, Incorprated, Gainesville, FL (US)

(72) Inventors: David Hammers, Gainesville, FL (US); Hugh Lee Sweeney, Gainesville, FL (US)

(73) Assignee: University of Florida Research Foundation, Incorprated, Gainesville, FL (US)

(21) Appl. No.: 18/286,453

(22) PCT Filed: Apr. 12, 2022
(86) PCT No.: PCT/US2022/024440

§ 371 Date: Oct. 11, 2023

Related U.S. Application Data
(60) Provisional application No. 63/173,982, filed on Apr. 12, 2021.

(51) Int. Cl. C07K 14/495; A61P 3/04; C07K 14/47; C12N 15/86

(52) U.S. Cl. CPC C07K 14/495; A61P 3/04; C07K 14/4716; C12N 15/86; C07K 2319/01; C12N 2750/14143

(57) ABSTRACT

The disclosure provides methods of treating obesity, reducing weight, or preventing weight gain using a combination therapy of GDF15 polypeptide and a myostatin inhibitor (e.g., a myostatin propeptide).

### RELATED APPLICATIONS

This Application claims priority under 35 U.S.C. § 119(e) to U.S. provisional patent application No. 63/173,982, filed Apr. 12, 2021, which is hereby incorporated by reference in its entirety.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 11, 2022, is named U119770193WO00-SEQ-GIC and is 66,470 bytes in size.

### BACKGROUND

Obesity is a major health problem in people and companion animals. Obesity increases the likelihood of development of various diseases, such as diabetes mellitus, hypertension, dyslipidemia, Type 2 diabetes, coronary heart disease, stroke, gallbladder disease, osteoarthritis, sleep apnea and breathing problems, certain types of cancer, and a low quality of life. Moreover, mortality risk directly correlates with obesity, such that, for example, a body-mass index in excess of 40 results in an average decreased life expectancy of more than 10 years.

None of the current modalities have been shown to effectively treat obesity without causing adverse effects, some of which can be very severe. For example, bariatric surgery is a major surgical procedure with a considerable risk of complications and requires extensive lifestyle modification. Drug therapy for obesity (e.g., using phentermine, phentermine-topiramate, lorcaserin, etc.) has limited efficacy and is further limited by side-effects.

In view of the prevalence and severity of obesity, along with the shortcomings of current treatment options, there is a need for alternative methods of treating obesity.

### SUMMARY

In one aspect, the present disclosure provides a method comprising administering to a subject one or more engineered nucleic acids encoding a (GDF15) polypeptide and a myostatin inhibitor.

In some embodiments, the method comprises administering to the subject a single engineered nucleic acid molecule comprising a nucleotide sequence encoding the GDF polypeptide and a nucleotide sequence comprising the myostatin inhibitor. In some embodiments, the engineered nucleic acid molecule comprises an internal ribosomal entry site (IRES) between the nucleotide sequence encoding the GDF15 polypeptide and the nucleotide sequence encoding the myostatin inhibitor.

In some embodiments, the method comprises administering to the subject a first engineered nucleic acid molecule comprising a nucleotide sequence encoding a growth differentiation factor 15 (GDF15) and a second engineered nucleic acid molecule comprising a nucleotide sequence encoding a myostatin inhibitor.

In some embodiments, the one or more engineered nucleic acid molecules are administered in amounts effective to reduce weight or prevent weight gain. In some embodiments, the one or more engineered nucleic acid molecules are administered in amounts effective to reduce weight or prevent weight gain without loss of muscle mass and/or loss of muscle function.

In one aspect, the disclosure provides a method comprising administering to a subject growth differentiation factor 15 (GDF15) polypeptide and a myostatin inhibitor.

In some embodiments, the GDF15 polypeptide and the myostatin inhibitor are present in the same composition. In some embodiments, the GDF15 polypeptide and the myostatin inhibitor are present in different compositions.

In some embodiments, the GDF15 polypeptide and the myostatin inhibitor are administered in amounts effective to reduce weight or prevent weight gain. In some embodiments, the GDF15 polypeptide and the myostatin inhibitor are administered in amounts effective to reduce weight or prevent weight gain without loss of muscle mass and/or loss of muscle function.

In one aspect, the present disclosure provides an engineered nucleic acid molecule comprising a nucleotide sequence encoding a growth differentiation factor (GDF15) polypeptide and a nucleotide sequence encoding a myostatin inhibitor. In some embodiments, the nucleic acid molecule comprises an internal ribosomal entry site (IRES) between the nucleotide sequence encoding the GDF15 polypeptide and the nucleotide sequence encoding the myostatin inhibitor.

In some embodiments, the GDF15 polypeptide comprises an amino acid sequence that is at least 80% identical to any one of SEQ ID NOs. 11-15 or to the mature peptide portion of any one of SEQ ID NOs. 11-15. In some embodiments, the GDF polypeptide is encoded by a nucleotide sequence that is at least 80% identical to the nucleotide sequence of any one of SEQ ID NOs. 26-30.

In some embodiments, the myostatin inhibitor is a myostatin propeptide. In some embodiments, the myostatin propeptide comprises an amino acid sequence that is at least 80% identical to any one of SEQ ID NOs. 1-10. In some embodiments, the myostatin propeptide comprises an amino acid sequence that is at least 80% identical to any one of SEQ ID NOs. 1-10, not including the signal sequence. In some embodiments, the myostatin propeptide comprises a mutation at the position corresponding to D76 in SEQ ID NO: 1. In some embodiments, the myostatin propeptide comprises an Ala at the position corresponding to D76 in SEQ ID NO: 1. In some embodiments, the myostatin propeptide is encoded by a nucleotide sequence that is at least 80% identical to the nucleotide sequence of any one of SEQ ID NOs. 16-25.

In some embodiments, the nucleotide sequence encoding the GDF polypeptide and/or the nucleotide sequence encoding the myostatin inhibitor are operably linked to a tissue-specific promoter. In some embodiments, the tissue-specific promoter is a liver-specific promoter or a skeletal-muscle-specific promoter.

In some embodiments, one or more nucleic acid molecules are present in one or more vectors. In some embodiments, at least one vector is a viral vector, optionally wherein the viral vector is an AAV vector.

In some embodiments, the one or more nucleic acid molecules are present in a lipid nanoparticle.

In one aspect, the present disclosure provides vectors, compositions, and cells comprising the engineered nucleic acids of the disclosure.

In one aspect, the present disclosure provides a method of expressing a GDF15 polypeptide and a myostatin inhibitor in a eukaryotic cell, comprising introducing an engineered nucleic acid molecule, a composition, or a vector of the disclosure into the eukaryotic cell.

In one aspect, the present disclosure provides a method of expressing a GDF15 polypeptide and a myostatin inhibitor in a subject, comprising administering to the subject an engineered nucleic acid molecule, a composition, or a vector of the disclosure.

In one aspect, the present disclosure provides a method of administering to a subject comprising administering to the subject an engineered nucleic acid molecule, a composition, or a vector of the disclosure. In some embodiments, the engineered nucleic acid molecule, composition, or vector are administered in amounts effective to reduce weight or prevent weight gain. In some embodiments, the engineered nucleic acid molecule, composition, or vector are administered in amounts effective to reduce weight or prevent weight gain without loss of muscle mass and/or loss of muscle function.

In some embodiments, the subject is an obese subject, an overweight subject, a subject having a family history of obesity, or a subject at risk of gaining weight. In some embodiments, the subject is a mammal. In some embodiments, the subject is a companion animal. In some embodiments, the companion animal is a dog, cat, or horse. In some embodiments, the subject is a human.

### BRIEF DESCRIPTION OF DRAWINGS

FIGS. 1A-1E show the effect of GDF15 and a protease-resistant mutant of the myostatin propeptide comprising the D76A mutation (dnMstn), singly or in combination, on body mass.

FIGS. 2A-2E show the effect of GDF15 and dnMstn, singly and in combination, on muscle mass.

FIGS. 3A and 3B show the effect of GDF15 and dnMstn, singly and in combination, on muscle function.

FIGS. 4A and 4B show an alignment of human, murine, canine, feline, and equine myostatin propeptides, and GDF proteins, respectively.

FIGS. 5A-5C show the effect of GDF15 and dnMstn, singly and in combination, on body weight, lean mass, and fat mass, respectively.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure.

The GDF15 pathway has been identified as a potential pathway for treating obesity. The loss of GDF15 is associated with weight gain and worsened metabolic function in mice. Xiong et al. (Sci Transl Med. 9(412):eaan8732 (2017)) demonstrated that treating mice with GDF15 improved metabolic health in mice, rats, and monkeys.

However, the inventors have found that, although GDF15 does decrease adipose tissue volume in a subject, it also leads to a severe loss of muscle mass, making it a poor therapeutic option. Without wishing to be bound by theory, the inventors have found that the mechanism by which GDF15 induces skeletal muscle loss is by stimulating myostatin, which is a negative regulator of muscle growth.

To counter the atrophic actions of GDF15, the inventors have combined GDF15 expression with the expression of an inhibitor of myostatin signaling (e.g., a myostatin propeptide). It is demonstrated herein that when a myostatin inhibitor (e.g., a myostatin propeptide) is secreted along with GDF15 into the blood of obese subjects, the obesity is resolved by GDF15 and the myostatin inhibitor blocks the atrophic actions of GDF15, thus maintaining muscle mass and/or muscle function. Thus, a combination therapy of GDF15 and a myostatin inhibitor (e.g., by secreting both GDF15 and a myostatin propeptide from an engineered nucleic acid) can be used to treat obesity in companion animals and humans.

Accordingly, the present disclosure relates to compositions and methods for treating obesity, reducing weight, or preventing weight gain, using a combination of GDF15 and a myostatin inhibitor (e.g., a myostatin propeptide).

In one aspect, the disclosure provides a method for treating obesity, reducing weight, or preventing weight gain, in a subject in need thereof, comprising administering to the subject one or more nucleic acid molecules encoding a GDF15 polypeptide and a myostatin inhibitor (e.g., myostatin propeptide).

In one aspect, the disclosure provides a method of treating obesity, reducing weight, or preventing weight gain, in a subject in need thereof, comprising administering to the subject effective amounts of a growth differentiation factor 15 (GDF15) polypeptide and a myostatin inhibitor (e.g., myostatin propeptide).

In one aspect, the disclosure provides an engineered nucleic acid molecule comprising a nucleotide sequence encoding a growth differentiation factor (GDF15) polypeptide and a nucleotide sequence encoding a myostatin inhibitor (e.g., myostatin propeptide).

GDF15 Polypeptides and Nucleic Acids. A GDF15 polypeptide includes any naturally occurring GDF15 polypeptide as well as any variants thereof (including mutants, truncations, fusions, and peptidomimetic forms) that retain a useful activity. GDF15 polypeptides include precursor forms expressed with a signal peptide, a proprotein form (containing both the prodomain and the mature portion), and the fully mature form. A GDF15 polypeptide may be a naturally occurring GDF15 protein from any species or a variant derived from such a protein by mutagenesis, truncation, or other modification. Variants may be selected to retain the ability to stimulate signaling by one or more of the known receptors of GDF15.

Non-limiting examples of native full-length GDF15 precursor proteins are described below.

Native mouse GDF15 precursor protein
is as follows
(SEQ ID NO: 11):
MAPPALQAQPPGGSQLSFLLFLLLLLLLLSWPSQGDALAMPEQRPSGPES
QLNADELRGRFQDLLSRLHANQSREDSNSEPSPDPAVRILSPEVRLGSHG
QLLLRVNRASLSQGLPEAYRVHRALLLLTPTARPWDITRPLKRALSLRGP
RAPALRLRLTPPPDLAMLPSGGTQLELRLRVAAGRGRRSAHAHPRDSCPL
GPGRCCHLETVQATLEDLGWSDWVLSPRQLQLSMCVGECPHLYRSANTHA
QIKARLHGLQPDKVPAPCCVPSSYTPVVLMHRTDSGVSLQTYDDLVARGC
HCA
Native human GDF15 precursor protein
is as follows
(SEQ ID NO: 12):
MPGQELRTVNGSQMLLVLLVLSWLPHGGALSLAEASRASFPGPSELHSED
SRFRELRKRYEDLLTRLRANQSWEDSNTDLVPAPAVRILTPEVRLGSGGH
LHLRISRAALPEGLPEASRLHRALFRLSPTASRSWDVTRPLRROLSLARP
QAPALHLRLSPPPSQSDQLLAESSSARPQLELHLRPQAARGRRRARARNG
DHCPLGPGRCCRLHTVRASLEDLGWADWVLSPREVQVTMCIGACPSQFRA
ANMHAQIKTSLHRLKPDTVPAPCCVPASYNPMVLIQKTDTGVSLQTYDDL
LAKDCHCI
Native canine GDF15 precursor protein
is as follows
(SEQ ID NO: 13):
MPGQGPAPAHCSPMLVILVMLSWLPSGGALSLAQEHLPAFPGPSDPHSST
DVSRIQELRKRYEHLQTKLRLNQGWADSNPDLVPATRVRILTPKLRLGPR
GHLHLRIARADLTAGLPAASRLHRALLRLSPTEPSSWDVTRPLORQLSRV
GSRTPTLRLRLLPRWDRSRALPSARPQLELHWRPRAARGRRNAHAHARDG
CPLGEGRCCRLQSLRASLQDLGWANWVVAPRELDVRMCVGACPSQFRSAN
THAQMQARLHGLNPDAAPAPCCVPASYEPVVLMHQDSDGRVSLTPFDDLV
AKDCHCV
Native feline GDF15 precursor protein
is as follows
(SEQ ID NO: 14):
MPGPGPTPPMLLMLLMLLMLCWLPSGGALSLAQEHLPAFPGPSEARSGTD
VSRFEEFRKLYEHLQTRLRLNQSWEDSNPDRVISEAQVRILIPKLRLGLG
GHLHLRIARADLTKGLPASFRLHRALLRLSPTELSSWDVTRPLRRQLSLG
GPGRPTLHLRLLPQRDRSPAALPSSARPQLELHWRPRAARGRRNAHARSK
DDCPLGAGRCCRLOSLRASLEDLGWASWVVAPRELDVRMCIGACPSQFRS
ANTHAQMQARLHGLNPDATPAPCCVPARYEPVVLMHQDSDGRVSLTPFDD
LVAKDCHCL
Native equine GDF15 precursor protein
is as follows
(SEQ ID NO: 15):
MRHFPMLLLSWLLLWLPPGGALPLTQDHSPAFLGPSGGHSSLDVSRFREL
RERYEHLQARLLLNQTQEDWNADPIPVDHVRMLTPKLRLGPDGHLRLHAL
RADLTEGLPAGSRLRQALLRLSPQAPGSWDLTRQLQRQLRLGGPAAPALS
LRLPRRGGPSPEALRAAQPRLELRWWPPAARGRRGAHAHARDACPLGEGR
CCRLLSLRASIEDLGWADWVVAPRELDVRMCVGACPGRFRSASRHAEAQA
RLHGLKPAAAPAPCCVPAGYAPVVLLHRAADGRVALTPFDDLVAAGCHCQ

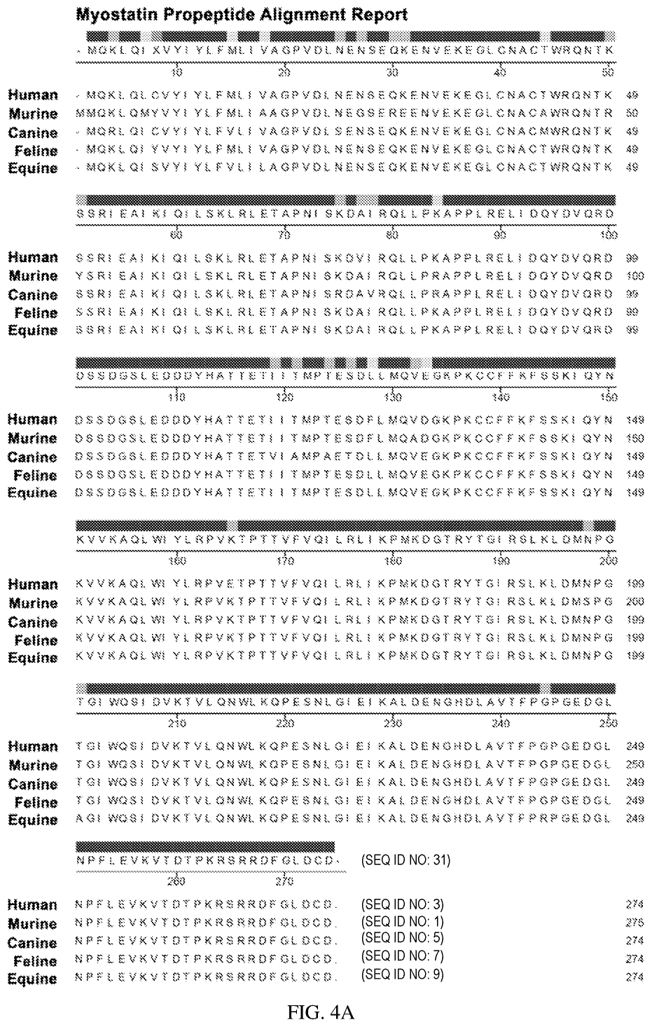

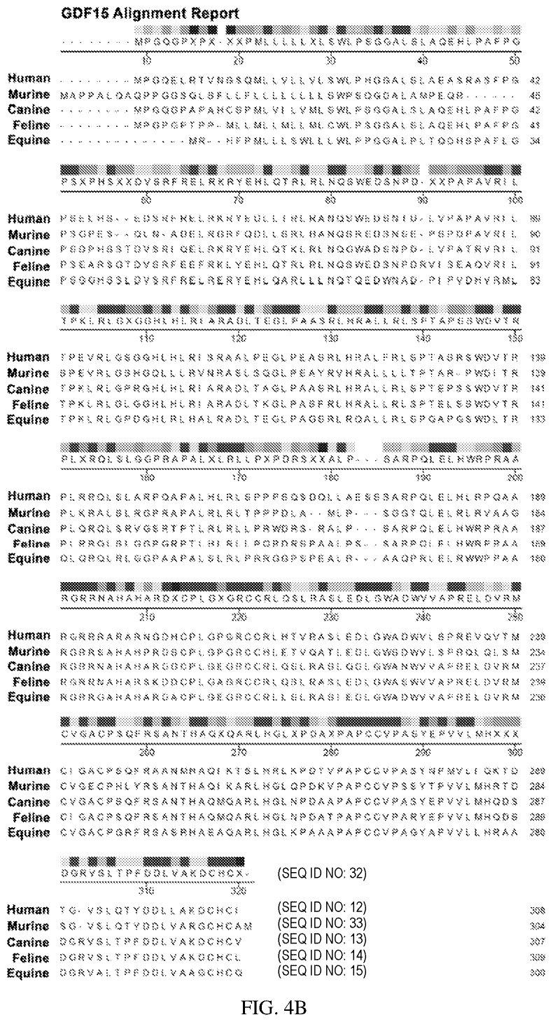

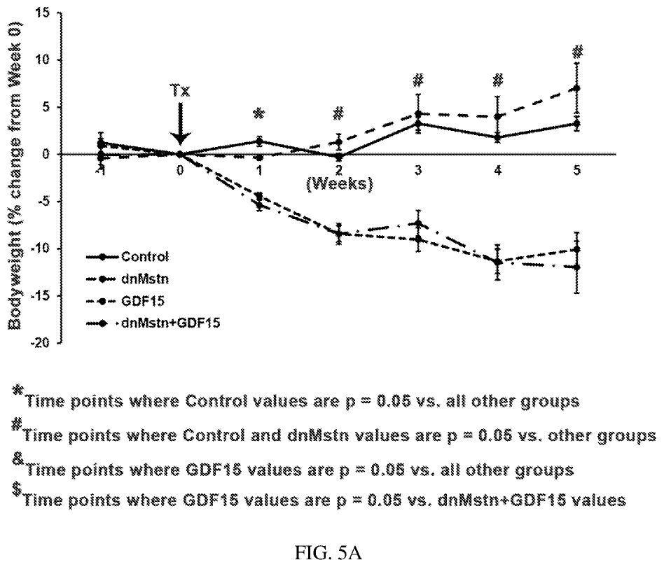

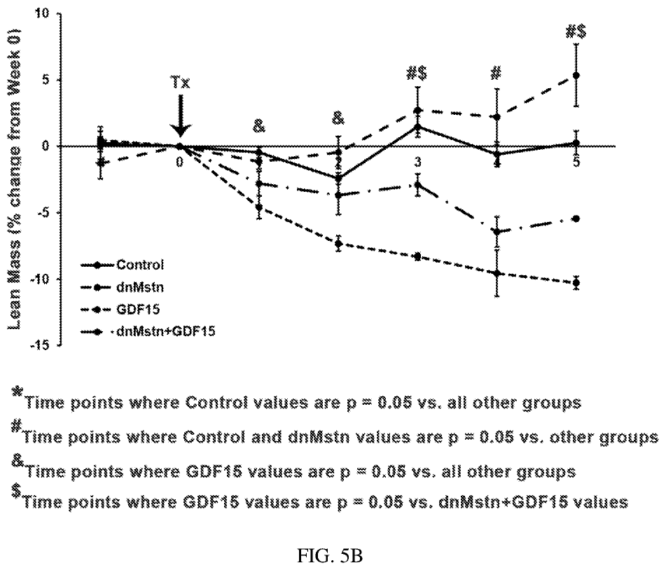

An alignment of the native human, murine, canine, feline and equine GDF precursor proteins is shown in FIG. 4B. A GDF15 polypeptide is further understood to include polypeptides that are variants of any one of SEQ ID NOs. 11-15. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID NOs. 11-15.

In some embodiments, a GDF polypeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 11, 12, 13, 14, and 15. In some embodiments, a GDF polypeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the mature peptide portion (e.g., without the signal sequence and/or the prodomain) of the amino acid sequence of any one of SEQ ID NOs. 11, 12, 13, 14, and 15.

In some embodiments, a GDF polypeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 11, 12, 13, 14, and 15. In some embodiments, a GDF polypeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the mature peptide portion (e.g., without the signal sequence and/or the prodomain) of the amino acid sequence of any one of SEQ ID NOs. 11, 12, 13, 14, and 15.

In some embodiments, a GDF polypeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 11, 12, 13, 14, and 15. In some embodiments, a GDF polypeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the mature peptide portion (e.g., without the signal sequence and/or the prodomain) of the amino acid sequence of any one of SEQ ID NOs. 11, 12, 13, 14, and 15.

In some embodiments, a GDF polypeptide comprises, consists essentially of, or consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 32, the consensus amino acid sequence for the human, murine, canine, feline, and equine GDF proteins, shown in FIG. 4B. The consensus sequence is provided below:

(SEQ ID NO: 32)
MPGQGPXPXXXPMLLLLLXLSWLPSGGALSLAQEHLPAFPGPSXPHSXXD
VSRFRELRKRYEHLQTRLRLNQSWEDSNPDXXPAPAVRILTPKLRLGXGG
HLHLRIARADLTEGLPAASRLHRALLRLSPTAPSSWDVTRPLXRQLSLGG
PRAPALXLRLLPXPDRSXXALPSARPQLELHWRPRAARGRRNAHAHARDX
CPLGXGRCCRLQSLRASLEDLGWADWVVAPRELDVRMCVGACPSQFRSAN
THAQXQARLHGLXPDAXPAPCCVPASYEPVVLMHXXXDGRVSLTPFDDLV
AKDCHCX.

In some embodiments, a GDF polypeptide comprises, consists essentially of, or consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 32. In some embodiments, a GDF polypeptide comprises, consists essentially of, or consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the mature peptide portion (e.g., without the signal sequence and/or the prodomain) of the amino acid sequence of SEQ ID NO: 32.

The signal peptide, propeptide, and mature peptide portions of GDF15 are known in the art and can be determined as such by one of skill in the art.

In some embodiments, functional variants or modified forms of GDF15 polypeptides include fusion proteins having at least a portion of the GDF15 polypeptide and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

In some aspects, the disclosure provides engineered nucleic acids encoding any of the GDF15 polypeptides described herein. Such nucleic acids may be DNA or RNA molecules. These nucleic acids may be used, for example, in methods for making GDF15 polypeptides or as direct therapeutic agents in a gene therapy approach. Non-limiting examples of nucleotide sequences encoding GDF polypeptides are as follows:

A codon-optimized nucleotide sequence encoding native
mouse GDF15 precursor protein is as follows
(SEQ ID NO: 26):
ATGGCCCCCCCCGCCCTGCAGGCCCAGCCCCCCGGCGGCAGCCAGCTGAGCTTCCTGCTGTTCC
TGCTGCTGCTGCTGCTGCTGCTGAGCTGGCCCAGCCAGGGCGACGCCCTGGCCATGCCCGAGCA
GCGGCCCAGCGGCCCCGAGAGCCAGCTGAACGCCGACGAGCTGCGGGGCCGGTTCCAGGACCTG
CTGAGCCGGCTGCACGCCAACCAGAGCCGGGAGGACAGCAACAGCGAGCCCAGCCCCGACCCCG
CCGTGCGGATCCTGAGCCCCGAGGTGCGGCTGGGCAGCCACGGCCAGCTGCTGCTGCGGGTGAA
CCGGGCCAGCCTGAGCCAGGGCCTGCCCGAGGCCTACCGGGTGCACCGGGCCCTGCTGCTGCTG
ACCCCCACCGCCCGGCCCTGGGACATCACCCGGCCCCTGAAGCGGGCCCTGAGCCTGCGGGGCC
CCCGGGCCCCCGCCCTGCGGCTGCGGCTGACCCCCCCCCCCGACCTGGCCATGCTGCCCAGCGG
CGGCACCCAGCTGGAGCTGCGGCTGCGGGTGGCCGCCGGCCGGGGCCGGCGGAGCGCCCACGCC
CACCCCCGGGACAGCTGCCCCCTGGGCCCCGGCCGGTGCTGCCACCTGGAGACCGTGCAGGCCA
CCCTGGAGGACCTGGGCTGGAGCGACTGGGTGCTGAGCCCCCGGCAGCTGCAGCTGAGCATGTG
CGTGGGCGAGTGCCCCCACCTGTACCGGAGCGCCAACACCCACGCCCAGATCAAGGCCCGGCTG
CACGGCCTGCAGCCCGACAAGGTGCCCGCCCCCTGCTGCGTGCCCAGCAGCTACACCCCCGTGG
TGCTGATGCACCGGACCGACAGCGGCGTGAGCCTGCAGACCTACGACGACCTGGTGGCCCGGGG
CTGCCACTGCGCCTGA
A codon-optimized nucleotide sequence encoding native
human GDF15 precursor protein is as follows
(SEQ ID NO: 27):
ATGCCCGGCCAGGAGCTGCGGACCGTGAACGGCAGCCAGATGCTGCTGGTGCTGCTGGTGCTGA
GCTGGCTGCCCCACGGCGGCGCCCTGAGCCTGGCCGAGGCCAGCCGGGCCAGCTTCCCCGGCCC
CAGCGAGCTGCACAGCGAGGACAGCCGGTTCCGGGAGCTGCGGAAGCGGTACGAGGACCTGCTG
ACCCGGCTGCGGGCCAACCAGAGCTGGGAGGACAGCAACACCGACCTGGTGCCCGCCCCCGCCG
TGCGGATCCTGACCCCCGAGGTGCGGCTGGGCAGCGGCGGCCACCTGCACCTGCGGATCAGCCG
GGCCGCCCTGCCCGAGGGCCTGCCCGAGGCCAGCCGGCTGCACCGGGCCCTGTTCCGGCTGAGC
CCCACCGCCAGCCGGAGCTGGGACGTGACCCGGCCCCTGCGGCGGCAGCTGAGCCTGGCCCGGC
CCCAGGCCCCCGCCCTGCACCTGCGGCTGAGCCCCCCCCCCAGCCAGAGCGACCAGCTGCTGGC
CGAGAGCAGCAGCGCCCGGCCCCAGCTGGAGCTGCACCTGCGGCCCCAGGCCGCCCGGGGCCGG
CGGCGGGCCCGGGCCCGGAACGGCGACCACTGCCCCCTGGGCCCCGGCCGGTGCTGCCGGCTGC
ACACCGTGCGGGCCAGCCTGGAGGACCTGGGCTGGGCCGACTGGGTGCTGAGCCCCCGGGAGGT
GCAGGTGACCATGTGCATCGGCGCCTGCCCCAGCCAGTTCCGGGCCGCCAACATGCACGCCCAG
ATCAAGACCAGCCTGCACCGGCTGAAGCCCGACACCGTGCCCGCCCCCTGCTGCGTGCCCGCCA
GCTACAACCCCATGGTGCTGATCCAGAAGACCGACACCGGCGTGAGCCTGCAGACCTACGACGA
CCTGCTGGCCAAGGACTGCCACTGCATC
A codon-optimized nucleotide sequence encoding native
canine GDF15 precursor protein is as follows
(SEQ ID NO: 28):
ATGCCCGGCCAGGGCCCCGCCCCCGCCCACTGCAGCCCCATGCTGGTGATCCTGGTGATGCTGA
GCTGGCTGCCCAGCGGCGGCGCCCTGAGCCTGGCCCAGGAGCACCTGCCCGCCTTCCCCGGCCC
CAGCGACCCCCACAGCAGCACCGACGTGAGCCGGATCCAGGAGCTGCGGAAGCGGTACGAGCAC
CTGCAGACCAAGCTGCGGCTGAACCAGGGCTGGGCCGACAGCAACCCCGACCTGGTGCCCGCCA
CCCGGGTGCGGATCCTGACCCCCAAGCTGCGGCTGGGCCCCCGGGGCCACCTGCACCTGCGGAT
CGCCCGGGCCGACCTGACCGCCGGCCTGCCCGCCGCCAGCCGGCTGCACCGGGCCCTGCTGCGG
CTGAGCCCCACCGAGCCCAGCAGCTGGGACGTGACCCGGCCCCTGCAGCGGCAGCTGAGCCGGG
TGGGCAGCCGGACCCCCACCCTGCGGCTGCGGCTGCTGCCCCGGTGGGACCGGAGCCGGGCCCT
GCCCAGCGCCCGGCCCCAGCTGGAGCTGCACTGGCGGCCCCGGGCCGCCCGGGGCCGGCGGAAC
GCCCACGCCCACGCCCGGGACGGCTGCCCCCTGGGCGAGGGCCGGTGCTGCCGGCTGCAGAGCC
TGCGGGCCAGCCTGCAGGACCTGGGCTGGGCCAACTGGGTGGTGGCCCCCCGGGAGCTGGACGT
GCGGATGTGCGTGGGCGCCTGCCCCAGCCAGTTCCGGAGCGCCAACACCCACGCCCAGATGCAG
GCCCGGCTGCACGGCCTGAACCCCGACGCCGCCCCCGCCCCCTGCTGCGTGCCCGCCAGCTACG
AGCCCGTGGTGCTGATGCACCAGGACAGCGACGGCCGGGTGAGCCTGACCCCCTTCGACGACCT
GGTGGCCAAGGACTGCCACTGCGTG
A codon-optimized nucleotide sequence encoding native
feline GDF15 precursor protein is as follows
(SEQ ID NO: 29):
ATGCCCGGCCCCGGCCCCACCCCCCCCATGCTGCTGATGCTGCTGATGCTGCTGATGCTGTGCT
GGCTGCCCAGCGGCGGCGCCCTGAGCCTGGCCCAGGAGCACCTGCCCGCCTTCCCCGGCCCCAG
CGAGGCCCGGAGCGGCACCGACGTGAGCCGGTTCGAGGAGTTCCGGAAGCTGTACGAGCACCTG
CAGACCCGGCTGCGGCTGAACCAGAGCTGGGAGGACAGCAACCCCGACCGGGTGATCAGCGAGG
CCCAGGTGCGGATCCTGACCCCCAAGCTGCGGCTGGGCCTGGGCGGCCACCTGCACCTGCGGAT
CGCCCGGGCCGACCTGACCAAGGGCCTGCCCGCCAGCTTCCGGCTGCACCGGGCCCTGCTGCGG
CTGAGCCCCACCGAGCTGAGCAGCTGGGACGTGACCCGGCCCCTGCGGCGGCAGCTGAGCCTGG
GCGGCCCCGGCCGGCCCACCCTGCACCTGCGGCTGCTGCCCCAGCGGGACCGGAGCCCCGCCGC
CCTGCCCAGCAGCGCCCGGCCCCAGCTGGAGCTGCACTGGCGGCCCCGGGCCGCCCGGGGCCGG
CGGAACGCCCACGCCCGGAGCAAGGACGACTGCCCCCTGGGCGCCGGCCGGTGCTGCCGGCTGC
AGAGCCTGCGGGCCAGCCTGGAGGACCTGGGCTGGGCCAGCTGGGTGGTGGCCCCCCGGGAGCT
GGACGTGCGGATGTGCATCGGCGCCTGCCCCAGCCAGTTCCGGAGCGCCAACACCCACGCCCAG
ATGCAGGCCCGGCTGCACGGCCTGAACCCCGACGCCACCCCCGCCCCCTGCTGCGTGCCCGCCC
GGTACGAGCCCGTGGTGCTGATGCACCAGGACAGCGACGGCCGGGTGAGCCTGACCCCCTTCGA
CGACCTGGTGGCCAAGGACTGCCACTGCCTG
A codon-optimized nucleotide sequence encoding native
equine GDF15 precursor protein is as follows
(SEQ ID NO: 30):
ATGCGGCACTTCCCCATGCTGCTGCTGAGCTGGCTGCTGCTGTGGCTGCCCCCCGGCGGCGCCC
TGCCCCTGACCCAGGACCACAGCCCCGCCTTCCTGGGCCCCAGCGGCGGCCACAGCAGCCTGGA
CGTGAGCCGGTTCCGGGAGCTGCGGGAGCGGTACGAGCACCTGCAGGCCCGGCTGCTGCTGAAC
CAGACCCAGGAGGACTGGAACGCCGACCCCATCCCCGTGGACCACGTGCGGATGCTGACCCCCA
AGCTGCGGCTGGGCCCCGACGGCCACCTGCGGCTGCACGCCCTGCGGGCCGACCTGACCGAGGG
CCTGCCCGCCGGCAGCCGGCTGCGGCAGGCCCTGCTGCGGCTGAGCCCCCAGGCCCCCGGCAGC
TGGGACCTGACCCGGCAGCTGCAGCGGCAGCTGCGGCTGGGCGGCCCCGCCGCCCCCGCCCTGA
GCCTGCGGCTGCCCCGGCGGGGCGGCCCCAGCCCCGAGGCCCTGCGGGCCGCCCAGCCCCGGCT
GGAGCTGCGGTGGTGGCCCCCCGCCGCCCGGGGCCGGCGGGGCGCCCACGCCCACGCCCGGGAC
GCCTGCCCCCTGGGCGAGGGCCGGTGCTGCCGGCTGCTGAGCCTGCGGGCCAGCATCGAGGACC
TGGGCTGGGCCGACTGGGTGGTGGCCCCCCGGGAGCTGGACGTGCGGATGTGCGTGGGCGCCTG
CCCCGGCCGGTTCCGGAGCGCCAGCCGGCACGCCGAGGCCCAGGCCCGGCTGCACGGCCTGAAG
CCCGCCGCCGCCCCCGCCCCCTGCTGCGTGCCCGCCGGCTACGCCCCCGTGGTGCTGCTGCACC
GGGCCGCCGACGGCCGGGTGGCCCTGACCCCCTTCGACGACCTGGTGGCCGCCGGCTGCCACTG
CCAG

A nucleotide sequence encoding a GDF15 polypeptide is further understood to include nucleotide sequences that are variants of any one of SEQ ID NOs. 26-30. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID NOs. 26-30.

In some embodiments, a GDF polypeptide is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 26, 27, 28, 29, or 30. In some embodiments, a GDF polypeptide is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the portion of the nucleotide sequence of any one of SEQ ID NOs. 26, 27, 28, 29, or 30 encoding the mature GDF15 peptide (e.g., without the signal sequence and/or the prodomain).

In some embodiments, a GDF polypeptide is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 26, 27, 28, 29, or 30. In some embodiments, a GDF polypeptide is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the portion of the nucleotide sequence of any one of SEQ ID NOs. 26, 27, 28, 29, or 30 encoding the mature GDF15 peptide (e.g., without the signal sequence and/or the prodomain).

In some embodiments, a GDF polypeptide is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 26, 27, 28, 29, or 30. In some embodiments, a GDF polypeptide is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 26, 27, 28, 29, or 30 encoding the mature GDF15 peptide.

It will be appreciated that the coding sequence of a nucleic acid does not include a stop codon. In some embodiments, any of the known stop codons may be used in a nucleic acid encoding a GDF polypeptide.

The “percent identity” of two amino acid sequences or nucleic acid sequences may be determined by any method known in the art.

In some embodiments, the percent identity of two nucleic acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST nucleotide searches can be performed with the NBLAST program, score=100, wordlength-12, to obtain guide sequences homologous to a target nucleic acid. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Myostatin Inhibitors. A myostatin inhibitor for use in the methods of the disclosure can be any known inhibitor of myostatin. In some embodiments, a myostatin inhibitor is an antibody. In some embodiments, a myostatin inhibitor is an interfering RNA. In some embodiments, a myostatin inhibitor is a myostatin propeptide. The myostatin propeptide is known to bind and inhibit myostatin.

A myostatin inhibitor may inhibit the expression, activity, and/or stability of myostatin. The term “inhibits” encompasses complete (100%) inhibition and partial (less than 100%) inhibition, otherwise referred to as reduction. Thus, a myostatin inhibitor may reduce, myostatin expression, stability, and/or activity by at least 10%, at least 20%, at least 30%, at least 40%, at least 50%, at least 60%, at least 70%, at least 80%, at least 90%, or at least 95%, relative to a control or baseline level. In some embodiments, the control or baseline level is the expression, stability, and/or activity of myostatin in the absence of the inhibitor.

Myostatin Propeptides and Nucleic Acids. A myostatin propeptide includes any naturally occurring myostatin propeptide as well as any variants thereof (including mutants, truncations, fusions, and peptidomimetic forms) that retain the ability to inhibit myostatin. A myostatin propeptide may be a naturally occurring myostatin propeptide from any species and variants derived from such propeptides by mutagenesis, truncation, or other modifications. Variants may be selected to retain the ability to inhibit myostatin activity.

Non-limiting examples of native myostatin propeptides are described below.

Native mouse myostatin propeptide is
as follows
(SEQ ID NO: 1):
MMQKLQMYVYIYLFMLIAAGPVDLNEGSEREENVEKEGLCNACAWRQNTR
YSRIEAIKIQILSKLRLETAPNISKDAIRQLLPRAPPLRELIDQYDVQRD
DSSDGSLEDDDYHATTETIITMPTESDELMQADGKPKCCFFKFSSKIQYN
KVVKAQLWIYLRPVKTPTTVFVQILRLIKPMKDGTRYTGIRSLKLDMSPG
IGIWQSIDVKTVLQNWLKQPESNLGIEIKALDENGHDLAVTFPGPGEDGL
NPFLEVKVTDTPKRSRRDFGLDCD
Native human myostatin propeptide is
as follows
(SEQ ID NO: 3):
MQKLQLCVYIYLFMLIVAGPVDLNENSEQKENVEKEGLCNACTWRQNTKS
SRIEAIKIQILSKLRLETAPNISKDVIRQLLPKAPPLRELIDQYDVQRDD
SSDGSLEDDDYHATTETIITMPTESDFLMQVDGKPKCCFFKFSSKIQYNK
VVKAQLWIYLRPVETPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVIDTPKRSRRDFGLDCD
Native canine myostatin propeptide is
as follows
(SEQ ID NO: 5):
MQRLQICVYIYLFVLIVAGPVDLSENSEQKENVEKEGLCNACMWRQNTKS
SRIEAIKIQILSKLRLETAPNISRDAVRQLLPRAPPLRELIDQYDVQRDD
SSDGSLEDDDYHATTETVIAMPAETDLLMQVEGKPKCCFFKFSSKIQYNK
VVKAQLWIYLRPVKTPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVTDTPKRSRRDFGLDCD
Native feline myostatin propeptide is
as follows
(SEQ ID NO: 7):
MQKLQIYVYIYLFMLIVAGPVDLNENSEQKENVEKEGLCNACTWRQNTKS
SRIEAIKIQILSKLRLETAPNISKDAIRQLLPKAPPLRELIDQYDVQRDD
SSDGSLEDDDYHATTETIITMPTESDLLMQVEGKPKCCFFKFSSKIQYNK
VVKAQLWIYLRPVKTPTTVFVQILRLIKPMKDGTRYTGIRSLKLDMNPGT
GIWQSIDVKIVLQNWLKQPESNLGIEIKALDENGHDLAVTFPGPGEDGLN
PFLEVKVTDTPKRSRRDFGLDCD
Native equine myostatin propeptide is
as follows
(SEQ ID NO: 9):
MQKLQISVYIYLFVLILAGPVDLNENSEQKENVEKEGLCNACTWRQNTKS
SRIEAIKIQILSKLRLETAPNISKDAIRQLLPKAPPLRELIDQYDVQRDD
SSDGSLEDDDYHATTETIITMPTESDLLMQVEGKPKCCFFKFSSKIQYNK
VVKAQLWIYLRPVKTPTTVFVQILRLIKPMKDGTRYTGIRSLKLDMNPGA
GIWQSIDVKTVLQNWLKQPESNLGIEIKALDENGHDLAVTFPRPGEDGLN
PFLEVKVTDTPKRSRRDFGLDCD

An alignment of the native human, murine, canine, feline and equine myostatin propeptides is shown in FIG. 4A. A myostatin propeptide is further understood to include polypeptides that are variants of any one of SEQ ID NOs. 1, 3, 5, 7, and 9. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID NOs. 1, 3, 5, 7, and 9.

In some embodiments, a myostatin propeptide does not include the signal sequence.

In some embodiments, a myostatin propeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 1, 3, 5, 7, and 9. In some embodiments, a myostatin propeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acid sequence of any one of SEQ ID NOs. 1, 3, 5, 7, and 9, not including the signal sequence.

In some embodiments, a myostatin propeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 1, 3, 5, 7, and 9. In some embodiments, a myostatin propeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 1, 3, 5, 7, and 9, not including the signal sequence.

In some embodiments, a myostatin propeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 11, 12, 13, 14, or 15. In some embodiments, a myostatin propeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 11, 12, 13, 14, or 15, not including the signal sequence.

In some embodiments, a myostatin propeptide comprises, consists essentially of, or consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 31, the consensus amino acid sequence for the human, murine, canine, feline, and equine myostatin propeptides, shown in FIG. 4A. The consensus sequence is provided below:

MQKLQIXVYIYLFMLIVAGPVDLNENSEQKENVEKEGLCNACTWRQNTKSSRIEAIKIQILSKL RLETAPNISKDAIRQLLPKAPPLRELIDQYDVQRDDSSDGSLEDDDYHATTETIITMPTESDLL MQVEGKPKCCFFKFSSKIQYNKVVKAQLWIYLRPVKTPTTVFVQILRLIKPMKDGTRYTGIRSL KLDMNPGTGIWQSIDVKTVLQNWLKQPESNLGIEIKALDENGHDLAVTFPGPGEDGLNPFLEVK VTDTPKRSRRDFGLDCD (SEQ ID NO: 31). In some embodiments, a myostatin propeptide comprises, consists essentially of, or consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 31, not including the signal sequence.

In some embodiments, a myostatin propeptide comprises a mutation (e.g., an amino acid substitution) at the position corresponding to D76 in SEQ ID NO: 1. In some embodiments, the myostatin propeptide comprises an Ala at the position corresponding to D76 in SEQ ID NO: 1.

Non-limiting examples of variant myostatin propeptides comprising an Ala at the position corresponding to D76 in SEQ ID NO: 1 are described below.

Mouse myostatin propeptide (D76A) is
as follows
(SEQ ID NO: 2):
MMQKLQMYVYIYLFMLIAAGPVDLNEGSEREENVEKEGLCNACAWRQNTR
YSRIEAIKIQILSKLRLETAPNISKAAIRQLLPRAPPLRELIDQYDVQRD
DSSDGSLEDDDYHATTETIITMPTESDELMQADGKPKCCFFKFSSKIQYN
KVVKAQLWIYLRPVKTPTTVFVQILRLIKPMKDGTRYTGIRSLKLDMSPG
IGIWQSIDVKTVLQNWLKQPESNLGIEIKALDENGHDLAVIFPGPGEDGL
NPFLEVKVTDTPKRSRRDFGLDCD
Human myostatin propeptide (D75A) is
as follows
(SEQ ID NO: 4):
MQKLQLCVYIYLFMLIVAGPVDLNENSEQKENVEKEGLCNACTWRQNTKS
SRIEAIKIQILSKLRLETAPNISKAVIRQLLPKAPPLRELIDQYDVQRDD
SSDGSLEDDDYHATTETIITMPTESDELMQVDGKPKCCFFKFSSKIQYNK
VVKAQLWIYLRPVETPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVIDTPKRSRRDFGLDCD
Canine myostatin propeptide (D75A) is
as follows
(SEQ ID NO: 6):
MQRLQICVYIYLFVLIVAGPVDLSENSEQKENVEKEGLCNACMWRQNTKS
SRIEAIKIQILSKLRLETAPNISRAAVRQLLPRAPPLRELIDQYDVQRDD
SSDGSLEDDDYHATTETVIAMPAETDLLMQVEGKPKCCFFKFSSKIQYNK
VVKAQLWIYLRPVKTPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVTDTPKRSRRDFGLDCD
Feline myostatin propeptide (D75A) is
as follows
(SEQ ID NO: 8):
MQKLQIYVYIYLFMLIVAGPVDLNENSEQKENVEKEGLCNACTWRQNTKS
SRIEAIKIQILSKLRLETAPNISKAAIRQLLPKAPPLRELIDQYDVQRDD
SSDGSLEDDDYHATTETIITMPTESDLLMQVEGKPKCCFFKFSSKIQYNK
VVKAQLWIYLRPVKTPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVTDTPKRSRRDFGLDCD
Equine myostatin propeptide (D75A) is
as follows
(SEQ ID NO: 10):
MQKLQISVYIYLFVLILAGPVDLNENSEQKENVEKEGLCNACTWRQNTKS
SRIEAIKIQILSKLRLETAPNISKAAIRQLLPKAPPLRELIDQYDVQRDD
SSDGSLEDDDYHATTETIITMPTESDLLMQVEGKPKCCFFKFSSKIQYNK
VVKAQLWIYLRPVKTPTTVFVQILRLIKPMKDGTRYTGIRSLKLDMNPGA
GIWQSIDVKTVLQNWLKQPESNLGIEIKALDENGHDLAVTFPRPGEDGLN
PFLEVKVTDTPKRSRRDFGLDCD

In some embodiments, a myostatin propeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10. In some embodiments, a myostatin propeptide comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10, not including the signal sequence.

In some embodiments, a myostatin propeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10. In some embodiments, a myostatin propeptide consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10, not including the signal sequence.

In some embodiments, a myostatin propeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10. In some embodiments, a myostatin propeptide consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs. 2, 4, 6, 8, and 10, not including the signal sequence.

In some embodiments, functional variants or modified forms of myostatin propeptides include fusion proteins having at least a portion of the myostatin propeptide and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

In some aspects, the disclosure provides engineered nucleic acids encoding any of the myostatin propeptides described herein. Such nucleic acids may be DNA or RNA molecules. These nucleic acids may be used, for example, in methods for making myostatin propeptides or as direct therapeutic agents in a gene therapy approach.

Non-limiting examples of nucleotide sequences encoding native myostatin propeptides are as follows:

A codon-optimized nucleotide sequence encoding native
mouse myostatin propeptide is as follows
(SEQ ID NO: 16):
ATGATGCAGAAGCTGCAGATGTACGTGTACATCTACCTGTTCATGCTGATCGCCGCCGGCCCCG
TGGACCTGAACGAGGGCAGCGAGCGGGAGGAGAACGTGGAGAAGGAGGGCCTGTGCAACGCCTG
CGCCTGGCGGCAGAACACCCGGTACAGCCGGATCGAGGCCATCAAGATCCAGATCCTGAGCAAG
CTGCGGCTGGAGACCGCCCCCAACATCAGCAAGGACGCCATCCGGCAGCTGCTGCCCCGGGCCC
CCCCCCTGCGGGAGCTGATCGACCAGTACGACGTGCAGCGGGACGACAGCAGCGACGGCAGCCT
GGAGGACGACGACTACCACGCCACCACCGAGACCATCATCACCATGCCCACCGAGAGCGACTTC
CTGATGCAGGCCGACGGCAAGCCCAAGTGCTGCTTCTTCAAGITCAGCAGCAAGATCCAGTACA
ACAAGGTGGTGAAGGCCCAGCTGTGGATCTACCTGCGGCCCGTGAAGACCCCCACCACCGTGTT
CGTGCAGATCCTGCGGCTGATCAAGCCCATGAAGGACGGCACCCGGTACACCGGCATCCGGAGC
CTGAAGCTGGACATGAGCCCCGGCACCGGCATCTGGCAGAGCATCGACGTGAAGACCGTGCTGC
AGAACTGGCTGAAGCAGCCCGAGAGCAACCTGGGCATCGAGATCAAGGCCCTGGACGAGAACGG
CCACGACCTGGCCGTGACCTTCCCCGGCCCCGGCGAGGACGGCCTGAACCCCTTCCTGGAGGTG
AAGGTGACCGACACCCCCAAGCGGAGCCGGCGGGACTTCGGCCTGGACTGCGACTGA
A codon-optimized nucleotide sequence encoding native
human myostatin propeptide is as follows
(SEQ ID NO: 18):
ATGCAGAAGCTGCAGCTGTGCGTGTACATCTACCTGTTCATGCTGATCGTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGACGTGATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACTTCCTG
ATGCAGGTGGACGGCAAGCCCAAGTGCTGCTTCTTCAAGTTCAGCAGCAAGATCCAGTACAACA
AGGTGGTGAAGGCCCAGCTGTGGATCTACCTGCGGCCCGTGGAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGGGGACTTCGGCCTGGACTGCGACTGA
A codon-optimized nucleotide sequence encoding native
canine myostatin propeptide is as follows
(SEQ ID NO: 20):
ATGCAGCGGCTGCAGATCTGCGTGTACATCTACCTGTTCGTGCTGATCGTGGCCGGCCCCGTGG
ACCTGAGCGAGAACAGCGAGCAGAAGGAGAACGTGGAGAAGGAGGGCCTGTGCAACGCCTGCAT
GTGGCGGCAGAACACCAAGAGCAGCCGGATCGAGGCCATCAAGATCCAGATCCTGAGCAAGCTG
CGGCTGGAGACCGCCCCCAACATCAGCCGGGACGCCGTGCGGCAGCTGCTGCCCCGGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCGTGATCGCCATGCCCGCCGAGACCGACCTGCTG
ATGCAGGTGGAGGGCAAGCCCAAGTGCTGCTTCTTCAAGTTCAGCAGCAAGATCCAGTACAACA
AGGTGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGCGGGACTICGGCCTGGACTGCGACTGA
A codon-optimized nucleotide sequence encoding native
feline myostatin propeptide is as follows
(SEQ ID NO: 22):
ATGCAGAAGCTGCAGATCTACGTGTACATCTACCTGTTCATGCTGATCGTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGACGCCATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACCTGCTG
ATGCAGGTGGAGGGCAAGCCCAAGTGCTGCTTCTTCAAGTTCAGCAGCAAGATCCAGTACAACA
AGGIGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGCGGGACTTCGGCCTGGACTGCGACTGA
A codon-optimized nucleotide sequence encoding native
equine myostatin propeptide is as follows
(SEQ ID NO: 24):
ATGCAGAAGCTGCAGATCAGCGTGTACATCTACCTGTTCGTGCTGATCCTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGACGCCATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACCTGCTG
ATGCAGGTGGAGGGCAAGCCCAAGTGCTGCTTCTTCAAGTTCAGCAGCAAGATCCAGTACAACA
AGGTGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCGCCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCCGGCCCGGCGAGGACGGCCTGAACCCCTTCCTGGAGGTGAAG
GTGACCGACACCCCCAAGCGGAGCCGGCGGGACTTCGGCCTGGACTGCGACTGA

Non-limiting examples of nucleotide sequences encoding variant myostatin propeptides comprising an Ala at the position corresponding to D76 in SEQ ID NO: lare as follows:

A codon-optimized nucleotide sequence encoding mouse
myostatin propeptide (D76A) is as follows
(SEQ ID NO: 17):
ATGATGCAGAAGCTGCAGATGTACGTGTACATCTACCTGTTCATGCTGATCGCCGCCGGCCCCG
TGGACCTGAACGAGGGCAGCGAGCGGGAGGAGAACGTGGAGAAGGAGGGCCTGTGCAACGCCTG
CGCCTGGCGGCAGAACACCCGGTACAGCCGGATCGAGGCCATCAAGATCCAGATCCTGAGCAAG
CTGCGGCTGGAGACCGCCCCCAACATCAGCAAGGCCGCCATCCGGCAGCTGCTGCCCCGGGCCC
CCCCCCTGCGGGAGCTGATCGACCAGTACGACGTGCAGCGGGACGACAGCAGCGACGGCAGCCT
GGAGGACGACGACTACCACGCCACCACCGAGACCATCATCACCATGCCCACCGAGAGCGACTTC
CTGATGCAGGCCGACGGCAAGCCCAAGTGCTGCTTCTTCAAGTTCAGCAGCAAGATCCAGTACA
ACAAGGTGGTGAAGGCCCAGCTGTGGATCTACCTGCGGCCCGTGAAGACCCCCACCACCGTGTT
CGTGCAGATCCTGCGGCTGATCAAGCCCATGAAGGACGGCACCCGGTACACCGGCATCCGGAGC
CTGAAGCTGGACATGAGCCCCGGCACCGGCATCTGGCAGAGCATCGACGTGAAGACCGTGCTGC
AGAACTGGCTGAAGCAGCCCGAGAGCAACCTGGGCATCGAGATCAAGGCCCTGGACGAGAACGG
CCACGACCTGGCCGTGACCTTCCCCGGCCCCGGCGAGGACGGCCTGAACCCCTTCCTGGAGGTG
AAGGTGACCGACACCCCCAAGCGGAGCCGGCGGGACTTCGGCCTGGACTGCGACTGA
A codon-optimized nucleotide sequence encoding human
myostatin propeptide (D75A) propeptide is as follows
(SEQ ID NO: 19):
ATGCAGAAGCTGCAGCTGTGCGTGTACATCTACCTGTTCATGCTGATCGTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGCCGTGATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACTTCCTG
ATGCAGGTGGACGGCAAGCCCAAGTGCTGCTTCTTCAAGTTCAGCAGCAAGATCCAGTACAACA
AGGTGGTGAAGGCCCAGCTGTGGATCTACCTGCGGCCCGTGGAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGCGGGACTTCGGCCTGGACTGCGACTGA
A codon-optimized nucleotide sequence encoding canine
myostatin propeptide (D75A) is as follows
(SEQ ID NO: 21):
ATGCAGCGGCTGCAGATCTGCGTGTACATCTACCTGTTCGTGCTGATCGTGGCCGGCCCCGTGG
ACCTGAGCGAGAACAGCGAGCAGAAGGAGAACGTGGAGAAGGAGGGCCTGTGCAACGCCTGCAT
GTGGCGGCAGAACACCAAGAGCAGCCGGATCGAGGCCATCAAGATCCAGATCCTGAGCAAGCTG
CGGCTGGAGACCGCCCCCAACATCAGCCGGGCCGCCGTGCGGCAGCTGCTGCCCCGGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCGTGATCGCCATGCCCGCCGAGACCGACCTGCTG
ATGCAGGTGGAGGGCAAGCCCAAGTGCTGCTTCTTCAAGTTCAGCAGCAAGATCCAGTACAACA
AGGTGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGCGGGACTTCGGCCTGGACTGCGACTGA
A codon-optimized nucleotide sequence encoding feline
myostatin propeptide (D75A) is as follows
(SEQ ID NO: 23):
ATGCAGAAGCTGCAGATCTACGTGTACATCTACCTGTTCATGCTGATCGTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGCCGCCATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACCTGCTG
ATGCAGGTGGAGGGCAAGCCCAAGTGCTGCTTCTTCAAGTTCAGCAGCAAGATCCAGTACAACA
AGGTGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCACCGGCATCTGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCGGCCCCGGCGAGGACGGCCTGAACCCCTTCCTGGAGGTGAAG
GTGACCGACACCCCCAAGCGGAGCCGGCGGGACTTCGGCCTGGACTGCGACTGA
A codon-optimized nucleotide sequence encoding equine
myostatin propeptide (D75A) is as follows
(SEQ ID NO: 25):
ATGCAGAAGCTGCAGATCAGCGTGTACATCTACCTGTTCGTGCTGATCCTGGCCGGCCCCGTGG
ACCTGAACGAGAACAGCGAGCAGAAGGAGAACGTGGAGAAGGAGGGCCTGTGCAACGCCTGCAC
CTGGCGGCAGAACACCAAGAGCAGCCGGATCGAGGCCATCAAGATCCAGATCCTGAGCAAGCTG
CGGCTGGAGACCGCCCCCAACATCAGCAAGGCCGCCATCCGGCAGCTGCTGCCCAAGGCCCCCC
CCCTGCGGGAGCTGATCGACCAGTACGACGTGCAGCGGGACGACAGCAGCGACGGCAGCCTGGA
GGACGACGACTACCACGCCACCACCGAGACCATCATCACCATGCCCACCGAGAGCGACCTGCTG
ATGCAGGTGGAGGGCAAGCCCAAGTGCTGCTTCTTCAAGTTCAGCAGCAAGATCCAGTACAACA
AGGTGGTGAAGGCCCAGCTGTGGATCTACCTGCGGCCCGTGAAGACCCCCACCACCGTGTTCGT
GCAGATCCTGCGGCTGATCAAGCCCATGAAGGACGGCACCCGGTACACCGGCATCCGGAGCCTG
AAGCTGGACATGAACCCCGGCGCCGGCATCIGGCAGAGCATCGACGTGAAGACCGTGCTGCAGA
ACTGGCTGAAGCAGCCCGAGAGCAACCTGGGCATCGAGATCAAGGCCCTGGACGAGAACGGCCA
CGACCTGGCCGTGACCTTCCCCCGGCCCGGCGAGGACGGCCTGAACCCCTTCCTGGAGGTGAAG
GTGACCGACACCCCCAAGCGGAGCCGGCGGGACTTCGGCCTGGACTGCGACTGA

A nucleotide sequence encoding a myostatin propeptide is further understood to include nucleotide sequences that are variants of any one of SEQ ID NOs. 16-25. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID NOs. 16-25.

In some embodiments, a myostatin propeptide is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25. In some embodiments, a myostatin propeptide is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25, not including the portion of the nucleotide sequence encoding the signal sequence.

In some embodiments, a myostatin propeptide is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25. In some embodiments, a myostatin propeptide is encoded by a nucleic acid consisting essentially of a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25, not including the portion of the nucleotide sequence encoding the signal sequence.

In some embodiments, a myostatin propeptide is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25. In some embodiments, a myostatin propeptide is encoded by a nucleic acid consisting of a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs. 16-25, not including the portion of the nucleotide sequence encoding the signal sequence.

It will be appreciated that the coding sequence of a nucleic acid does not include a stop codon. In some embodiments, any of the known stop codons can be used in nucleic acids encoding a myostatin propeptide.

Engineered Nucleic Acids. In some aspects, the present disclosure provides one or more engineered nucleic acid molecules encoding a GDF polypeptide and/or a myostatin inhibitor (e.g., myostatin propeptide).

In some embodiments, the present disclosure provides separate nucleic acid molecules comprising a nucleotide sequence encoding a growth differentiation factor 15 (GDF15) and a nucleotide sequence comprising the myostatin inhibitor (e.g., myostatin propeptide).

In some embodiments, the present disclosure provides a single nucleic acid molecule comprising a nucleotide sequence encoding a growth differentiation factor 15 (GDF15) and a nucleotide sequence comprising the myostatin inhibitor (e.g., myostatin propeptide). This allows the combined secretion of both GDF15 and a myostatin inhibitor (e.g., myostatin propeptide).

In some embodiments, the GDF polypeptide and the myostatin inhibitor (e.g., myostatin propeptide) are operably linked to a single promoter. In some embodiments, the GDF polypeptide and the myostatin inhibitor (e.g., myostatin propeptide) are under the control of separate promoters.

In some embodiments, the engineered nucleic acid molecule comprises an internal ribosomal entry site (IRES) between the nucleotide sequence encoding the GDF15 polypeptide and the nucleotide sequence encoding the myostatin inhibitor (e.g., myostatin propeptide). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the GDF15 polypeptide is translated first and the myostatin inhibitor (e.g., myostatin propeptide) is translated second. In some embodiments, the myostatin inhibitor (e.g., myostatin propeptide) is translated first and the GDF15 polypeptide is translated second. Alternative means of bicistronic expression (e.g., 2A peptides) known in the art are also contemplated herein.

An engineered nucleic acid or nucleic acid molecule (e.g., at least two nucleotides covalently linked together, and in some instances, containing phosphodiester bonds, referred to as a phosphodiester backbone) is a nucleic acid that does not occur in nature. The engineered nucleic acids include recombinant nucleic acids and synthetic nucleic acids. In some embodiments, a recombinant nucleic acid is a molecule that is constructed by joining nucleic acids (e.g., isolated nucleic acids, synthetic nucleic acids or a combination thereof) from a single source or multiple sources. A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903,2010, each of which is incorporated by reference herein). GIBSON ASSEMBLY® typically uses three enzymatic activities in a single-tube reaction: 5′ exonuclease, the 3′extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

Expression of the GDF15 polypeptide and/or the myostatin inhibitor (e.g., myostatin propeptide) may be controlled using one or more regulatory sequences such as enhancers and promoters, operably linked to the nucleotide sequences encoding the GDF15 polypeptide and/or the myostatin inhibitor (e.g., myostatin propeptide). In some embodiments the nucleotide sequences encoding the GDF polypeptide and the myostatin inhibitor (e.g., myostatin propeptide) are operably controlled by a single promoter. In other embodiments, the nucleotide sequence encoding the GDF polypeptide and the nucleotide sequence encoding the myostatin inhibitor (e.g., myostatin propeptide) are operably controlled by distinct promoters.

A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence.

Promoters that may be used in accordance with the present disclosure may comprise any suitable promoter that can drive the expression of the nucleotide sequences encoding the GDF polypeptide and the myostatin inhibitor (e.g., myostatin propeptide).

In some embodiments, a promoter may be one that is naturally associated with the gene, and may be obtained by isolating the 5′ non-coding sequence upstream of the coding segment and/or exon of a given gene. Such a promoter may be referred to as an endogenous promoter or a native promoter.

In some embodiments, the promoter may be a chimeric promoter comprising sequence elements from two or more different promoters.

In some embodiments, the promoter may be a tissue-specific promoter. A “tissue-specific promoter”, as used herein, refers to promoters that preferentially or selectively function in a specific type of tissue, e.g., liver, muscle (e.g., skeletal muscle), heart, etc. In some embodiments, a tissue-specific promoter is not able to drive the expression of the genes in other types of tissues. In some embodiments, the promoter that may be used in accordance with the present disclosure is a liver-specific promoter. Non-limiting examples of liver-specific promoters include the human serum albumin promoter, the hybrid liver promoter (HLP), hepatic control region (HCR)-human a-1 antitrypsin (hAAT) promoter, liver fatty acid binding protein promoter, SV40/bAlb promoter, SV40/halb promoter. In some embodiments, the liver-specific promoter is the hAAT promoter. In some embodiments, the promoter that may be used in accordance with the present disclosure is a muscle-specific (e.g., skeletal muscle-specific) promoter. Non-limiting examples of muscle-specific (e.g., skeletal muscle-specific) include skeletal α-actin promoter, desmin promoter, dMCK promoter, tMCK promoter, CK6 promoter, CK8 promoter, or Mb promoter. In some embodiments, a promoter is a skeletal muscle-specific promoter. In some embodiments, the skeletal muscle-specific promoter is the CK6 promoter, CK8 promoter, or skeletal α-actin promoter.

In some embodiments, the promoter is a constitutively active promoter. Constitutive promoters include any constitutive promoter described herein or known to one of ordinary skill in the art. Non-limiting examples of constitutive promoters include the immediate early cytomegalovirus (CMV) promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as mammalian gene promoters such as, but not limited to, the elongation Factor-la (EF-1a)the actin promoter, the myosin promoter, the hemoglobin promoter, and the creatine kinase promoter.

Inducible promoters are also contemplated herein. An “inducible promoter” refers to a promoter that is characterized by regulating (e.g., initiating or activating) transcriptional activity when in the presence of, influenced by or contacted by an inducer signal.

In some embodiments, the engineered nucleic acids of the present disclosure further comprise one or more enhancer elements. In some embodiments, an enhancer element is an ApoF enhancer. In some embodiments, the promoter is a liver-specific promoter (e.g., a hAAT promoter) with ApoF enhancer elements.

In some embodiments, the engineered nucleic acids of the present disclosure further comprise additional regulatory sequences, including, without limitation, a 3′ untranslated region (3′UTR), and/or a poly-adenylation (polyA) signal sequence.

Vectors. In some aspects, the present disclosure provides vectors comprising the engineered nucleic acids described herein. In some embodiments, the GDF15 polypeptide and the myostatin inhibitor (e.g., a myostatin propeptide) are expressed from a single vector. In some embodiments, the GDF15 polypeptide and the myostatin inhibitor (e.g., a myostatin propeptide) are expressed from different vectors. A vector is any nucleic acid that may be used as a vehicle to deliver exogenous (foreign) genetic material to a cell. A vector, in some embodiments, is a DNA sequence that includes an insert (e.g., a nucleotide sequence encoding a GDF15 polypeptide or a nucleotide sequence encoding a myostatin inhibitor (e.g., myostatin propeptide)) and a larger sequence that serves as the backbone of the vector. Non-limiting examples of vectors include plasmids, viruses/viral vectors, phagemids, cosmids (comprising a plasmid and Lambda phage cos sequences), and artificial chromosomes, any of which may be used as provided herein. In some embodiments, the vector is a viral vector, such as a viral particle. In some embodiments, the viral vector is an adenovirus, adeno associated virus (AAV), γ-retrovirus, HSV, lentivirus, or Sendai virus vector. In some embodiments, the viral vector is an AAV vector.

In some embodiments, a nucleic acid of the disclosure is flanked by AAV ITRs for packaging into an AAV vector.

The phrase AAV vector can include a recombinant AAV (rAAV) genome comprising the gene(s) of interest flanked by AAV ITRs, and an rAAV particle comprising an rAAV genome encapsidated with rAAV capsid proteins.

In some embodiments, the GDF15 polypeptide and the myostatin inhibitor (e.g., a myostatin propeptide) are expressed from a single promoter (e.g., via a bicistronic mRNA that encodes both proteins). In some embodiments, the GDF15 polypeptide and the myostatin inhibitor (e.g., a myostatin propeptide) are expressed from different promoters in the same rAAV vector. In some embodiments, the GDF15 polypeptide and the myostatin inhibitor (e.g., a myostatin propeptide) are expressed from different promoters in different rAAV vectors.

In some aspects, the methods described herein comprise expressing a GDF15 and/or a myostatin inhibitor (e.g., myostatin propeptide) in cells. The vectors provided herein may be used for gene therapy for treating obesity, reducing weight, or preventing weight gain in a subject in need thereof.

Methods of Use. In some aspects, the present disclosure provides methods of treating obesity, reducing weight, or preventing weight gain in a subject in need thereof.

In some aspects, the present disclosure provides methods of administering a growth differentiation factor 15 (GDF15) polypeptide and a myostatin inhibitor (e.g., a myostatin propeptide) to a subject. In some aspects, the present disclosure provides methods of administering one or more nucleic acid molecules encoding a GDF15 polypeptide and a myostatin inhibitor (e.g., myostatin propeptide) to a subject. In some embodiments, the subject is an obese subject, an overweight subject, a subject having a family history of obesity, or a subject at risk of gaining weight.

In one aspect, the present disclosure provides a method of treating obesity, reducing weight, or preventing weight gain in a subject in need thereof, comprising administering to the subject effective amounts of a growth differentiation factor 15 (GDF15) polypeptide and a myostatin inhibitor (e.g., a myostatin propeptide). In some embodiments, the GDF15 polypeptide is a mature GDF15 peptide. In some embodiments, the GDF15 polypeptide is a full-length GDF15 precursor protein. In some embodiments, the myostatin propeptide does not include the signal sequence.

In another aspect, the present disclosure provides a gene therapy for treating obesity, reducing weight, or preventing weight gain in a subject in need thereof. Accordingly, the present disclosure provides a method of treating obesity, reducing weight, or preventing weight gain, in a subject in need thereof, comprising administering to the subject an effective amount of one or more nucleic acid molecules encoding a GDF15 polypeptide and a myostatin inhibitor (e.g., myostatin propeptide). In some embodiments, the one or more nucleic acid molecules encode a full-length GDF15 precursor protein. In some embodiments, the one or more nucleic acid molecules encode a mature GDF15 protein. In some embodiments, the one or more nucleic acid molecules encode a myostatin propeptide with the signal sequence. In some embodiments, the one or more nucleic acid molecules encode a myostatin propeptide without the signal sequence.

The present disclosure thus contemplates methods of expressing a GDF15 polypeptide in combination with an myostatin inhibitor (e.g., myostatin propeptide) in a subject for treating obesity, reducing weight, or preventing weight gain, the method comprising administering to a subject in need thereof an effective amount of one or more engineered nucleic acids of the disclosure.

In some embodiments, the methods of the disclosure promote loss of fat mass.

In some embodiments, the methods of the disclosure maintain muscle mass. In some embodiments, the methods of the disclosure maintain muscle function. Maintenance of muscle mass or muscle function is achieved when the muscle mass or muscle function are with 5%, 6%, 7%, 8%, 9%, 10%, 15%, 20%, 25%, or 30% of a baseline or control level. In some embodiments, the baseline or control level is the muscle mass or muscle function prior to treatment.

In some embodiments, the methods of the disclosure inhibit loss of muscle mass. In some embodiments, the methods of the disclosure inhibit loss of muscle function. The term “inhibits” encompasses complete (100%) inhibition and partial (less than 100%) inhibition, otherwise referred to as reduction. Thus, the methods of the disclosure may reduce loss of muscle mass by at least 10%, at least 20%, at least 30%, at least 40%, at least 50%, at least 60%, at least 70%, at least 80%, at least 90%, or at least 95%, relative to a control. In some embodiments, the control level is the level of loss of muscle mass or muscle function in the absence of the myostatin inhibitor (e.g., myostatin propeptide).

The terms “subject,” and “patient,” are used interchangeably herein. In some embodiments, a subject is a mammal, such as a human, a nonhuman primate, a dog, a cat, a horse, a sheep, a poultry, a cow, a pig, a mouse, a rat, a rodent, or a goat. In some embodiments, the subject and mammal is a human. In some embodiments, the subject is a companion animal. In some embodiments, the companion animal is a dog, cat, or horse. In some embodiments, the subject is an obese subject, an overweight subject, a subject having a family history of obesity, or a subject at risk of gaining weight. In some embodiments, a subject is a human subject 50 years or older, 55 years or older, 60 years or older, 65 years or older, 70 years or older, 75 years or older, or 80 years or older. In some embodiments, a subject is a dog 8 years or older, 9 years or older, 10 years or older, 12 years or older, or 13 years or older. In some embodiments, a subject is a cat 10 years or older, 11 years or older, 12 years or older, 13 years or older, 14 year or older, or 15 years or older. In some embodiments, an older subject is an older horse (e.g., 15 years or older, 20 years or older, 25 years or older, 30 years or older, 35 years or older, or 40 years or older).

An “effective amount” of the compositions of the disclosure generally refers to an amount sufficient to elicit the desired biological response, e.g., express the GDF15 polypeptide and the myostatin inhibitor (e.g., myostatin propeptide) in a target cell, treat obesity, reduce weight, prevent weight gain, etc. In some embodiments, an effective amount is an amount required to reduce body mass in a subject. In some embodiments, an effective amount is an amount required to reduce body mass while maintaining muscle mass and/or muscle function (e.g., without loss of muscle mass and/or muscle function). In some embodiments, an effective amount is an amount required to reduce body mass while inhibiting loss of muscle mass and/or muscle function. As will be appreciated by those of ordinary skill in this art, the effective amount of an agent described herein may vary depending on such factors as the condition being treated, the mode of administration, and the age, body composition, and health of the subject. Suitable dosage ranges are readily determinable by one skilled in the art.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

Compositions. In some aspects, the present disclosure provides compositions comprising the polypeptides, engineered nucleic acids, or vectors disclosed herein. For administration to a subject, the polypeptides, engineered nucleic acids, or vectors disclosed herein may be formulated in a composition. In some embodiments, the composition further comprises additional agents (e.g., for specific delivery, increasing half-life, or other therapeutic agents).

In some embodiments, the composition is a pharmaceutical composition. In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Engineered nucleic acids or polypeptides, in some embodiments, may be formulated in a non-viral delivery vehicle. In some embodiments, a nucleic acid encoding the GDF15 polypeptide may be delivered by a vector (e.g., a viral vector such as an rAAV vector) while a nucleic acid encoding the myostatin inhibitor (e.g., a myostatin propeptide), or the myostatin inhibitor itself, is delivered by a non-viral delivery vehicle, or vice versa. Non-limiting examples of non-viral delivery vehicles include nanoparticles, such as nanocapsules and nanospheres. See, e.g., Sing, R et al. Exp Mol Pathol. 2009; 86(3):215-223. A nanocapsule is often comprised of a polymeric shell encapsulating an agent. Nanospheres are often comprised of a solid polymeric matrix throughout which the agent is dispersed. In some embodiments, the nanoparticle is a lipid particle, such as a liposome. See, e.g., Puri, A et al. Crit Rev Ther Drug Carrier Syst. 2009; 26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283,1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the engineered nucleic acids disclosed herein may be found, for example, in Remington's Pharmaceutical Sciences, 18th edition, Mack Publishing Co., Easton, Pa (1990) (incorporated herein by reference in its entirety).

Methods of Administration. Any of the polypeptides, engineered nucleic acids, vectors, or compositions disclosed herein may be administered to a subject to treat obesity, reduce weight, or prevent weight gain.

Suitable routes of administration include, without limitation, intravenous, intranasal, intramuscular, intrathecal, or subcutaneous. In some embodiments, a polypeptide, engineered nucleic acid, vector, or composition of the disclosure is administered intravenously, subcutaneously, intramuscularly intrathecally or intranasally. In some embodiments, a polypeptide, engineered nucleic acid, vector, or composition of the disclosure is administered directly (e.g., by direct injection) to one or more cells (e.g., liver cells or skeletal muscle cells), tissues (e.g., skeletal muscle), or organs (e.g., liver). In some embodiments, a polypeptide, engineered nucleic acid, vector, or composition of the disclosure is administered directly (e.g., by direct injection) to the liver. Other routes of administration are contemplated herein. The administration route can be changed depending on a number of factors, including the desired cell, tissue, or organ.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intraarticular, and intramuscular administration and formulation.

In some embodiments, the nucleic acids of the disclosure are delivered via an AAV vector. In some embodiments, the number of AAV particles administered to a subject may be on the order ranging from about 10⁶ to about 2×10¹⁴ particles/mL or about 10³ to about 10¹³ particles/mL, or any values in between for either range, such as for example, about 10⁶, 10⁷, 10⁸, 10⁹, 10¹⁰, 10¹¹, 10¹², 10¹³, or 10¹⁴ particles/mL. In some embodiments, the number of AAV particles administered to a subject may be on the order ranging from about 10⁶ to about 2×10¹⁴ vector genomes(vgs)/mL or 10³ to 10¹⁵ vgs/mL, or any values in between for either range, such as for example, about 10⁶, 10⁷, 10⁸, 10⁹, 10¹⁰, 10¹¹, 10¹², 10¹³, or 10¹⁴ or 10¹⁵ vgs/mL. The AAV particles can be administered as a single dose, or divided into two or more administrations as may be required to achieve therapy.

In some embodiments, a single engineered nucleic acid encodes a GDF15 polypeptide and a myostatin inhibitor (e.g., myostatin propeptide) and the engineered nucleic acid or a composition or vector comprising the engineered nucleic acid is administered to the subject. In some embodiments, a GDF15 polypeptide and a myostatin propeptide or a nucleic acid encoding a GDF15 polypeptide and a nucleic acid encoding a myostatin inhibitor (e.g., myostatin propeptide) may be administered in separate compositions or vectors. The GDF15 polypeptide (or the nucleic acid encoding the GDF15 polypeptide) and the myostatin inhibitor (e.g., myostatin propeptide) (or the nucleic acid encoding the myostatin propeptide) are administered close enough in time to beneficially affect the treatment. In some embodiments, the GDF15 polypeptide (or the nucleic acid encoding the GDF15 polypeptide) is administered separately in time from or simultaneously with the myostatin inhibitor (e.g., myostatin propeptide) (or the nucleic acid encoding the myostatin inhibitor (e.g., myostatin propeptide)).

### EXAMPLES

### Example 1. GDF15 and Myostatin Propeptide Gene Therapy to Treat Obesity

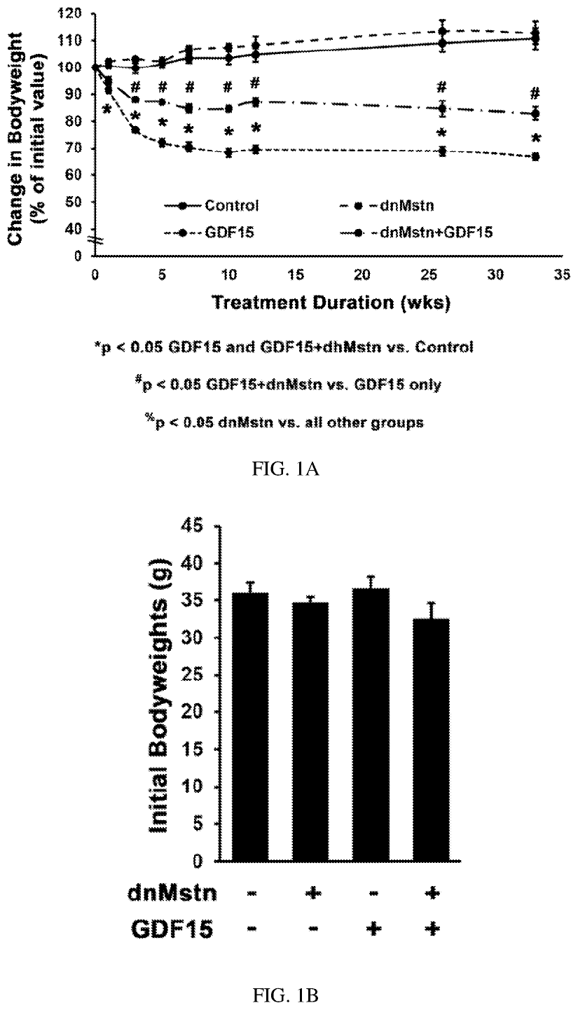

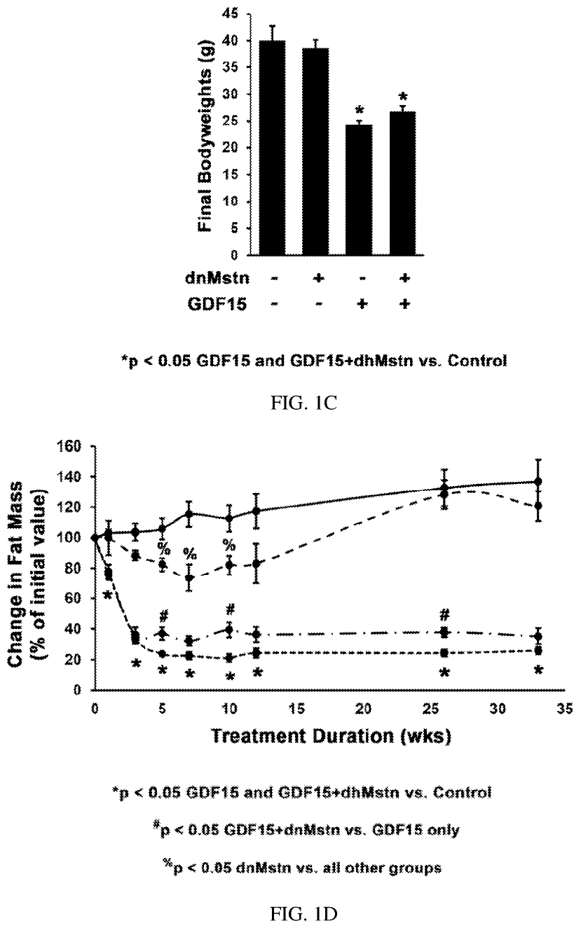

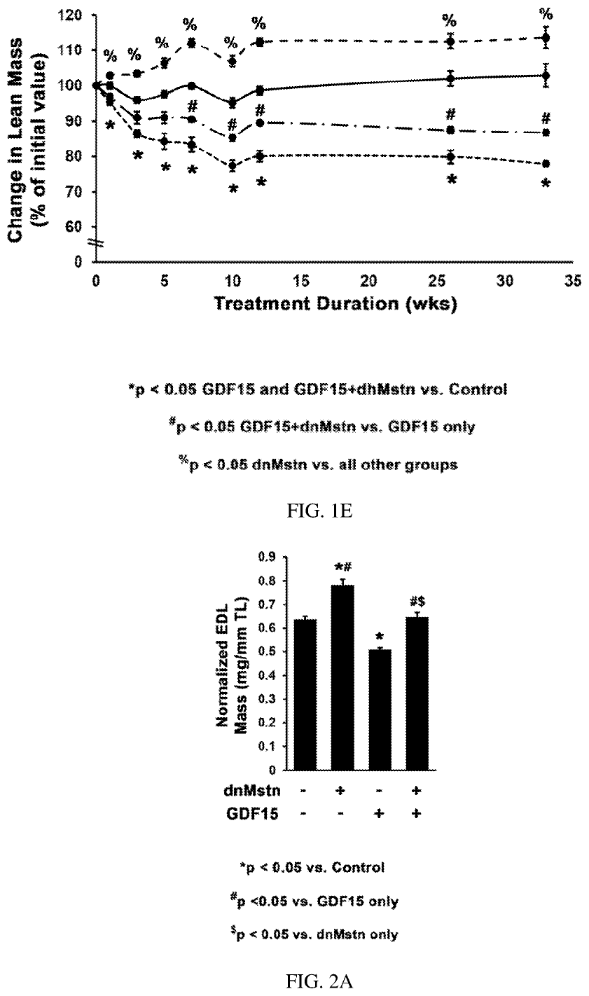

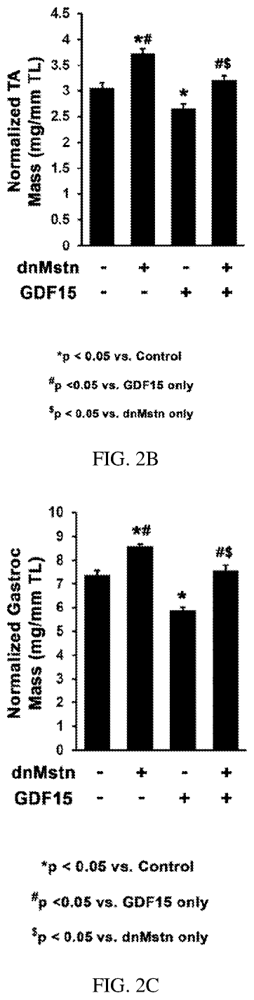

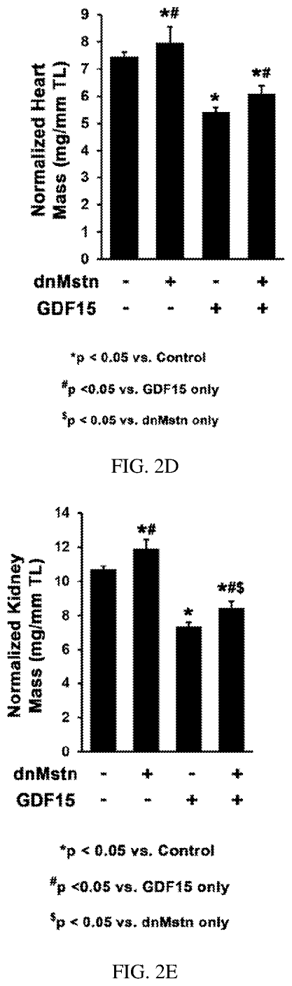

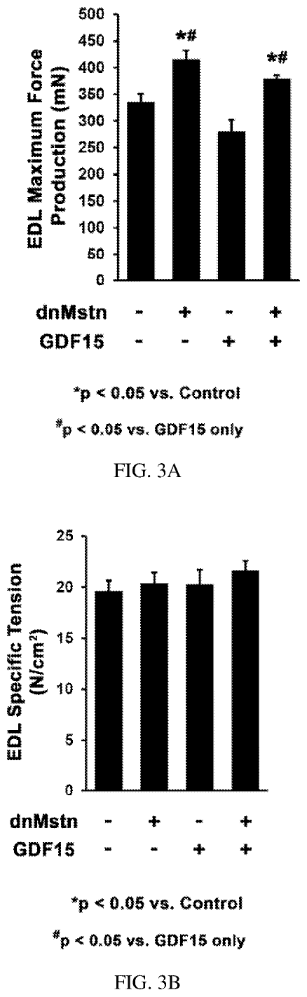

GDF15 or dnMstn, a protease-resistant myostatin propeptide comprising the D76A mutation, were expressed singly or in combination in mice. To obtain secretion of both GDF15 and dnMstn, a bi-cistronic RNA was created using an IRES, with GDF15 being translated first, followed by myostatin. The construct was designed as codon-optimized dnMstn-IRES-co-optimized GDF14. Over a treatment period of over 30 weeks, changes in body weight and body composition, as well changes in muscle mass and muscle function were evaluated. Decreases in body weight, fat mass, and lean mass were observed in mice that received GDF15 gene therapy alone and mice that received a combination gene therapy of GDF15 and dnMstn (FIGS. 1A-1E). However, mice that received GDF15 gene therapy alone also had concomitant reductions in muscle mass and muscle function, while muscle mass and muscle function were maintained in mice that received a combination gene therapy of GDF15 and dnMstn. Muscle mass data obtained for extensor digitorum longus (EDL), gastrocnemis, tibialis anterior (TA), heart, and kidney muscles are shown in FIGS. 2A-2E. Muscle function data obtained for EDL muscle as determined by measuring the maximum force and specific tension, is shown in FIGS. 3A-3B.

Thus, dnMstn is able to counter the atrophic actions of GDF15. A combination of GDF15 and dnMstn leads to a reduction in bodyweight, fat mass, and lean mass, while maintaining muscle mass and muscle function. Accordingly, a combination therapy of GDF15 and myostatin propeptide is useful to treat obesity.

### Example 2. GDF15 and Myostatin Propeptide Gene Therapy in Older Mice

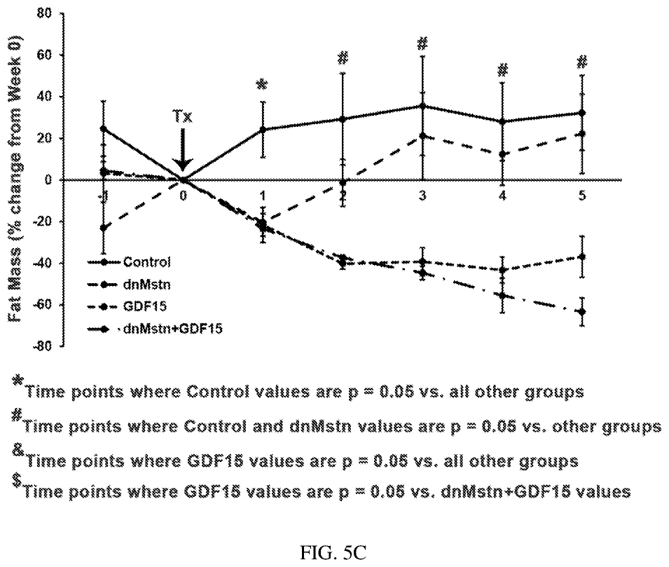

Twenty-one week-old C57BL/6 mice received control, dnMstn, GDF15, or dnMstn+GDF15 treatments at Week 0. Body masses and compositions were followed for 5 weeks to determine longitudinal changes during this time frame. Body weights were significantly and indistinguishably decreased by GDF15 and dnMstn+GDF15 treatments, compared to the control and dnMstn groups (FIG. 5A). Lean body mass is significantly better maintained in the dnMstn+GDF15 group compared to treatment with GDF15 alone, indicating attenuation of muscle mass loss (FIG. 5B), whereas loss of fat mass is unchanged between these groups (FIG. 5C). These data indicate that the loss of bodyweight and fat mass, as well as the sparing of muscle mass, incurred by the dnMstn+GDF15 treatment occurs in the context of aging.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A method comprising administering to a subject one or more engineered nucleic acids encoding a (GDF15) polypeptide and a myostatin inhibitor.

2. The method of claim 1, comprising administering to the subject a single engineered nucleic acid molecule comprising a nucleotide sequence encoding the GDF polypeptide and a nucleotide sequence comprising the myostatin inhibitor.

3. The method of claim 2, wherein the engineered nucleic acid molecule comprises an internal ribosomal entry site (IRES) between the nucleotide sequence encoding the GDF15 polypeptide and the nucleotide sequence encoding the myostatin inhibitor.

4. The method of claim 1, comprising administering to the subject a first engineered nucleic acid molecule comprising a nucleotide sequence encoding a growth differentiation factor 15 (GDF15) and a second engineered nucleic acid molecule comprising a nucleotide sequence encoding a myostatin inhibitor.

5. The method of any one of claims 1-4, wherein the one or more engineered nucleic acid molecules are administered in amounts effective to reduce weight or prevent weight gain.

6. The method of any one of claims 1-5, wherein the one or more engineered nucleic acid molecules are administered in amounts effective to reduce weight or prevent weight gain without loss of muscle mass and/or loss of muscle function.

7. The method of any one of claims 1-6, wherein the GDF15 polypeptide comprises an amino acid sequence that is at least 80% identical to any one of SEQ ID NOs. 11-15.

8. The method of any one of claims 1-7, wherein the myostatin inhibitor is a myostatin propeptide.

9. The method of claim 8, wherein the myostatin propeptide comprises an amino acid sequence that is at least 80% identical to any one of SEQ ID NOs. 1-10.

10. The method of 8 or 9, wherein the myostatin propeptide comprises a mutation at the position corresponding to D76 in SEQ ID NO: 1.

11. The method of claim 10, wherein the myostatin propeptide comprises an Ala at the position corresponding to D76 in SEQ ID NO: 1.

12. The method of any one of claims 1-11, wherein the GDF polypeptide is encoded by a nucleotide sequence that is at least 80% identical to the nucleotide sequence of any one of SEQ ID NOs. 26-30.

13. The method of any one of claims 8-12, wherein the myostatin propeptide is encoded by a nucleotide sequence that is at least 80% identical to the nucleotide sequence of any one of SEQ ID NOs. 16-25.

14. The method of any one of claims 1-13, wherein the nucleotide sequence encoding the GDF polypeptide and/or the nucleotide sequence encoding the myostatin inhibitor are operably linked to a tissue-specific promoter.

15. The method of claim 14, wherein the tissue-specific promoter is a liver-specific promoter or a skeletal-muscle-specific promoter.

16. The method of any one of claims 1-15, wherein the one or more nucleic acid molecules are present in one or more vectors.

17. The method of claim 16, wherein at least one vector is a viral vector, optionally wherein the viral vector is an AAV vector.

18. The method of any one of claims 1-15, wherein the one or more nucleic acid molecules are present in a lipid nanoparticle.

19. A method comprising administering to a subject growth differentiation factor 15 (GDF15) polypeptide and a myostatin inhibitor.

20. The method of claim 19, wherein the GDF15 polypeptide and the myostatin inhibitor are present in the same composition.

21. The method of claim 20, wherein the GDF15 polypeptide and the myostatin inhibitor are present in different compositions.

22. The method of any one of claims 19-21, wherein the GDF15 polypeptide and the myostatin inhibitor are administered in amounts effective to reduce weight or prevent weight gain.

23. The method of any one of claims 19-22, wherein the GDF15 polypeptide and the myostatin inhibitor are administered in amounts effective to reduce weight or prevent weight gain without loss of muscle mass and/or loss of muscle function.

24. The method of any one of claims 19-23, wherein the GDF15 polypeptide comprises an amino acid sequence that is at least 80% identical to the amino acid sequence of any one of SEQ ID NOs. 11-15.

25. The method of any one of claim 19-24, wherein the myostatin inhibitor is a myostatin propeptide.

26. The method of claim 25, wherein the myostatin propeptide comprises an amino acid sequence that is at least 80% identical to the amino acid sequence of any one of SEQ ID NOs. 1-10.

27. The method of claim 25 or 26, wherein the myostatin propeptide comprises a mutation at the position corresponding to D76 in SEQ ID NO: 1.

28. The method of claim 27, wherein the myostatin propeptide comprises an Ala at the position corresponding to D76 in SEQ ID NO: 1.

29. The method of any one of claims 1-28, wherein the subject is an obese subject, an overweight subject, a subject having a family history of obesity, or a subject at risk of gaining weight.

30. The method of any one of claims 1-29, wherein the subject is a mammal.

31. The method of claim 30, wherein the subject is a companion animal.

32. The method of claim 31, wherein the companion animal is a dog, cat, or horse.

33. The method of claim 32, wherein the subject is a human.

34. An engineered nucleic acid molecule comprising a nucleotide sequence encoding a growth differentiation factor (GDF15) polypeptide and a nucleotide sequence encoding a myostatin inhibitor.

35. The engineered nucleic acid molecule of claim 34, wherein the nucleic acid molecule comprises an internal ribosomal entry site (IRES) between the nucleotide sequence encoding the GDF15 polypeptide and the nucleotide sequence encoding the myostatin inhibitor.

36. The engineered nucleic acid molecule of claim 34, wherein the GDF15 polypeptide comprises an amino acid sequence that is at least 80% identical to the amino acid sequence of any one of SEQ ID NOs. 11-15.

37. The engineered nucleic acid molecule of any one of claims 34-36, wherein the myostatin inhibitor is a myostatin propeptide.

38. The engineered nucleic acid molecule of claim 37, wherein the myostatin propeptide comprises an amino acid sequence that is at least 80% identical to the amino acid sequence of any one of SEQ ID NOs. 1-10.

39. The engineered nucleic acid molecule of claim 37 or 38, wherein the myostatin propeptide comprises a mutation at the position corresponding to D76 in SEQ ID NO: 1.

40. The engineered nucleic acid molecule of claim 39, wherein the myostatin propeptide comprises an Ala at the position corresponding to D76 in SEQ ID NO: 1.

41. The engineered nucleic acid molecule of any one of claims 34-40, wherein the nucleotide sequence encoding the GDF15 polypeptide comprises a nucleotide sequence that is at least 80% identical to any one of SEQ ID NOs. 26-30.

42. The engineered nucleic acid molecule of any one of claims 37-41, wherein the nucleotide sequence encoding the myostatin propeptide comprises a nucleotide sequence that is at least 80% identical to any one of SEQ ID NOs. 16-25.

43. The engineered nucleic acid molecule of any one of claims 34-42, wherein a tissue-specific promoter is operably linked to the nucleotide sequence encoding the GDF polypeptide and/or the nucleotide sequence encoding the myostatin inhibitor.

44. The engineered nucleic acid molecule of claim 43, wherein the tissue-specific promoter is a liver-specific promoter or a skeletal-muscle-specific promoter.

45. A composition comprising the engineered nucleic acid of any one of claims 34-44.

46. A vector comprising the engineered nucleic acid of any one of claims 34-44.

47. The vector of claim 46, wherein the vector is a viral vector, optionally an AAV vector.

48. An engineered cell comprising the engineered nucleic acid molecule of any one of claims 34-44 or the vector of claim 46 or 47.

49. A method of expressing a GDF15 polypeptide and a myostatin inhibitor in a eukaryotic cell, comprising introducing the engineered nucleic acid molecule of any one of claims 34-44, the composition of claim 45, or the vector of claim 46 or 47 into the eukaryotic cell.

50. A method of expressing a GDF15 polypeptide and a myostatin inhibitor in a subject, comprising administering to the subject the engineered nucleic acid molecule of any one of claims 34-44, the composition of claim 45, or the vector of claim 46 or 47.

51. A method comprising administering to a subject an engineered nucleic acid molecule of any one of claims 34-44, the composition of claim 45, or the vector of claim 46 or 47.

52. The method of claim 51, wherein the one or more engineered nucleic acid molecules, composition, or vector are administered in amounts effective to reduce weight or prevent weight gain.

53. The method of claim 51 or 52, wherein the one or more engineered nucleic acid molecules, composition, or vector are administered in amounts effective to reduce weight or prevent weight gain without loss of muscle mass and/or loss of muscle function.

54. The method of any one of claims 50-53, wherein the subject is an obese subject, an overweight subject, a subject having a family history of obesity, or a subject at risk of gaining weight.

55. The method of any one of claims 54, wherein the subject is a mammal.

56. The method of claim 55, wherein the subject is a companion animal.

57. The method of claim 56, wherein the companion animal is a dog, cat, or horse.

58. The method of claim 55, wherein the subject is a human.


SEQUENCE LISTING (33 sequences)

SEQ ID NO: 1 - protein, 274 aa, Unknown
  Murine
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCD

SEQ ID NO: 2 - protein, 274 aa, Artificial Sequence
  Synthetic
MMQKLQMYVYIYLFMLIAAGPVDLNEGSEREENVEKEGLCNACAWRQNTRYSRIEAIKIQILSKLRLETAPNISKAAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCD

SEQ ID NO: 3 - protein, 273 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 4 - protein, 273 aa, Artificial Sequence
  Synthetic
MQKLQLCVYIYLFMLIVAGPVDLNENSEQKENVEKEGLCNACTWRQNTKSSRIEAIKIQILSKLRLETAPNISKAVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 5 - protein, 273 aa, Unknown
  Canine
MQRLQICVYIYLFVLIVAGPVDLSENSEQKENVEKEGLCNACMWRQNTKSSRIEAIKIQILSKLRLETAPNISRDAVRQLLPRAPPLRELIDQYDVQRDDSSDGSLEDDDYHATTETVIAMPAETDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 6 - protein, 273 aa, Artificial Sequence
  Synthetic
MQRLQICVYIYLFVLIVAGPVDLSENSEQKENVEKEGLCNACMWRQNTKSSRIEAIKIQILSKLRLETAPNISRAAVRQLLPRAPPLRELIDQYDVQRDDSSDGSLEDDDYHATTETVIAMPAETDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 7 - protein, 273 aa, Unknown
  Feline
MQKLQIYVYIYLFMLIVAGPVDLNENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 8 - protein, 273 aa, Artificial Sequence
  Synthetic
MQKLQIYVYIYLFMLIVAGPVDLNENSEQKENVEKEGLCNACTWRQNTKSSRIEAIKIQILSKLRLETAPNISKAAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 9 - protein, 273 aa, Unknown
  Equine
MQKLQISVYIYLFVLILAGPVDLNENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGAGIWQSIDVKTVLQNWLKQPESNLGIEIKALDENGHDLAVTFPRPGEDGLNPFLEVKVTDTPKRSRRDFGLDCD

SEQ ID NO: 10 - protein, 273 aa, Artificial Sequence
  Synthetic
MQKLQISVYIYLFVLILAGPVDLNENSEQKENVEKEGLCNACTWRQNTKSSRIEAIKIQILSKLRLETAPNISKAAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGAGIWQSIDVKTVLQNWLKQPESNLGIEIKALDENGHDLAVTFPRPGEDGLNPFLEVKVTDTPKRSRRDFGLDCD

SEQ ID NO: 11 - protein, 303 aa, Unknown
  Murine
MAPPALQAQPPGGSQLSFLLFLLLLLLLLSWPSQGDALAMPEQRPSGPESQLNADELRGRFQDLLSRLHANQSREDSNSEPSPDPAVRILSPEVRLGSHGQLLLRVNRASLSQGLPEAYRVHRALLLLTPTARPWDITRPLKRALSLRGPRAPALRLRLTPPPDLAMLPSGGTQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCA

SEQ ID NO: 12 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 13 - protein, 307 aa, Unknown
  Canine
MPGQGPAPAHCSPMLVILVMLSWLPSGGALSLAQEHLPAFPGPSDPHSSTDVSRIQELRKRYEHLQTKLRLNQGWADSNPDLVPATRVRILTPKLRLGPRGHLHLRIARADLTAGLPAASRLHRALLRLSPTEPSSWDVTRPLQRQLSRVGSRTPTLRLRLLPRWDRSRALPSARPQLELHWRPRAARGRRNAHAHARDGCPLGEGRCCRLQSLRASLQDLGWANWVVAPRELDVRMCVGACPSQFRSANTHAQMQARLHGLNPDAAPAPCCVPASYEPVVLMHQDSDGRVSLTPFDDLVAKDCHCV

SEQ ID NO: 14 - protein, 309 aa, Unknown
  Feline
MPGPGPTPPMLLMLLMLLMLCWLPSGGALSLAQEHLPAFPGPSEARSGTDVSRFEEFRKLYEHLQTRLRLNQSWEDSNPDRVISEAQVRILTPKLRLGLGGHLHLRIARADLTKGLPASFRLHRALLRLSPTELSSWDVTRPLRRQLSLGGPGRPTLHLRLLPQRDRSPAALPSSARPQLELHWRPRAARGRRNAHARSKDDCPLGAGRCCRLQSLRASLEDLGWASWVVAPRELDVRMCIGACPSQFRSANTHAQMQARLHGLNPDATPAPCCVPARYEPVVLMHQDSDGRVSLTPFDDLVAKDCHCL

SEQ ID NO: 15 - protein, 300 aa, Unknown
  Equine
MRHFPMLLLSWLLLWLPPGGALPLTQDHSPAFLGPSGGHSSLDVSRFRELRERYEHLQARLLLNQTQEDWNADPIPVDHVRMLTPKLRLGPDGHLRLHALRADLTEGLPAGSRLRQALLRLSPQAPGSWDLTRQLQRQLRLGGPAAPALSLRLPRRGGPSPEALRAAQPRLELRWWPPAARGRRGAHAHARDACPLGEGRCCRLLSLRASIEDLGWADWVVAPRELDVRMCVGACPGRFRSASRHAEAQARLHGLKPAAAPAPCCVPAGYAPVVLLHRAADGRVALTPFDDLVAAGCHCQ

SEQ ID NO: 16 - DNA, 825 bp, Artificial Sequence
  Synthetic
atgatgcagaagctgcagatgtacgtgtacatctacctgttcatgctgatcgccgccggccccgtggacctgaacgagggcagcgagcgggaggagaacgtggagaaggagggcctgtgcaacgcctgcgcctggcggcagaacacccggtacagccggatcgaggccatcaagatccagatcctgagcaagctgcggctggagaccgcccccaacatcagcaaggacgccatccggcagctgctgccccgggccccccccctgcgggagctgatcgaccagtacgacgtgcagcgggacgacagcagcgacggcagcctggaggacgacgactaccacgccaccaccgagaccatcatcaccatgcccaccgagagcgacttcctgatgcaggccgacggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgagccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 17 - DNA, 825 bp, Artificial Sequence
  Synthetic
atgatgcagaagctgcagatgtacgtgtacatctacctgttcatgctgatcgccgccggccccgtggacctgaacgagggcagcgagcgggaggagaacgtggagaaggagggcctgtgcaacgcctgcgcctggcggcagaacacccggtacagccggatcgaggccatcaagatccagatcctgagcaagctgcggctggagaccgcccccaacatcagcaaggccgccatccggcagctgctgccccgggccccccccctgcgggagctgatcgaccagtacgacgtgcagcgggacgacagcagcgacggcagcctggaggacgacgactaccacgccaccaccgagaccatcatcaccatgcccaccgagagcgacttcctgatgcaggccgacggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgagccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 18 - DNA, 822 bp, Artificial Sequence
  Synthetic
atgcagaagctgcagctgtgcgtgtacatctacctgttcatgctgatcgtggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggacgtgatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacttcctgatgcaggtggacggcaagcccaagtgctgcttcttcaagttcagcagcaagatccagtacaacaaggtggtgaaggcccagctgtggatctacctgcggcccgtggagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 19 - DNA, 822 bp, Artificial Sequence
  Synthetic
atgcagaagctgcagctgtgcgtgtacatctacctgttcatgctgatcgtggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggccgtgatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacttcctgatgcaggtggacggcaagcccaagtgctgcttcttcaagttcagcagcaagatccagtacaacaaggtggtgaaggcccagctgtggatctacctgcggcccgtggagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 20 - DNA, 822 bp, Artificial Sequence
  Synthetic
atgcagcggctgcagatctgcgtgtacatctacctgttcgtgctgatcgtggccggccccgtggacctgagcgagaacagcgagcagaaggagaacgtggagaaggagggcctgtgcaacgcctgcatgtggcggcagaacaccaagagcagccggatcgaggccatcaagatccagatcctgagcaagctgcggctggagaccgcccccaacatcagccgggacgccgtgcggcagctgctgccccgggccccccccctgcgggagctgatcgaccagtacgacgtgcagcgggacgacagcagcgacggcagcctggaggacgacgactaccacgccaccaccgagaccgtgatcgccatgcccgccgagaccgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 21 - DNA, 822 bp, Artificial Sequence
  Synthetic
atgcagcggctgcagatctgcgtgtacatctacctgttcgtgctgatcgtggccggccccgtggacctgagcgagaacagcgagcagaaggagaacgtggagaaggagggcctgtgcaacgcctgcatgtggcggcagaacaccaagagcagccggatcgaggccatcaagatccagatcctgagcaagctgcggctggagaccgcccccaacatcagccgggccgccgtgcggcagctgctgccccgggccccccccctgcgggagctgatcgaccagtacgacgtgcagcgggacgacagcagcgacggcagcctggaggacgacgactaccacgccaccaccgagaccgtgatcgccatgcccgccgagaccgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 22 - DNA, 822 bp, Artificial Sequence
  Synthetic
atgcagaagctgcagatctacgtgtacatctacctgttcatgctgatcgtggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggacgccatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 23 - DNA, 822 bp, Artificial Sequence
  Synthetic
atgcagaagctgcagatctacgtgtacatctacctgttcatgctgatcgtggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggccgccatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcaccggcatctggcagagcatcgacgtgaagaccgtgctgcagaactggctgaagcagcccgagagcaacctgggcatcgagatcaaggccctggacgagaacggccacgacctggccgtgaccttccccggccccggcgaggacggcctgaaccccttcctggaggtgaaggtgaccgacacccccaagcggagccggcgggacttcggcctggactgcgactga

SEQ ID NO: 24 - DNA, 822 bp, Artificial Sequence
  Synthetic
atgcagaagctgcagatcagcgtgtacatctacctgttcgtgctgatcctggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggacgccatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcgccggcatctggcagagcatcgacgtgaagaccgtgctgcagaactggctgaagcagcccgagagcaacctgggcatcgagatcaaggccctggacgagaacggccacgacctggccgtgaccttcccccggcccggcgaggacggcctgaaccccttcctggaggtgaaggtgaccgacacccccaagcggagccggcgggacttcggcctggactgcgactga

SEQ ID NO: 25 - DNA, 822 bp, Artificial Sequence
  Synthetic
atgcagaagctgcagatcagcgtgtacatctacctgttcgtgctgatcctggccggccccgtggacctgaacgagaacagcgagcagaaggagaacgtggagaaggagggcctgtgcaacgcctgcacctggcggcagaacaccaagagcagccggatcgaggccatcaagatccagatcctgagcaagctgcggctggagaccgcccccaacatcagcaaggccgccatccggcagctgctgcccaaggccccccccctgcgggagctgatcgaccagtacgacgtgcagcgggacgacagcagcgacggcagcctggaggacgacgactaccacgccaccaccgagaccatcatcaccatgcccaccgagagcgacctgctgatgcaggtggagggcaagcccaagtgctgcttcttcaagttcagcagcaagatccagtacaacaaggtggtgaaggcccagctgtggatctacctgcggcccgtgaagacccccaccaccgtgttcgtgcagatcctgcggctgatcaagcccatgaaggacggcacccggtacaccggcatccggagcctgaagctggacatgaaccccggcgccggcatctggcagagcatcgacgtgaagaccgtgctgcagaactggctgaagcagcccgagagcaacctgggcatcgagatcaaggccctggacgagaacggccacgacctggccgtgaccttcccccggcccggcgaggacggcctgaaccccttcctggaggtgaaggtgaccgacacccccaagcggagccggcgggacttcggcctggactgcgactga

SEQ ID NO: 26 - DNA, 912 bp, Artificial Sequence
  Synthetic
atggccccccccgccctgcaggcccagccccccggcggcagccagctgagcttcctgctgttcctgctgctgctgctgctgctgctgagctggcccagccagggcgacgccctggccatgcccgagcagcggcccagcggccccgagagccagctgaacgccgacgagctgcggggccggttccaggacctgctgagccggctgcacgccaaccagagccgggaggacagcaacagcgagcccagccccgaccccgccgtgcggatcctgagccccgaggtgcggctgggcagccacggccagctgctgctgcgggtgaaccgggccagcctgagccagggcctgcccgaggcctaccgggtgcaccgggccctgctgctgctgacccccaccgcccggccctgggacatcacccggcccctgaagcgggccctgagcctgcggggcccccgggcccccgccctgcggctgcggctgacccccccccccgacctggccatgctgcccagcggcggcacccagctggagctgcggctgcgggtggccgccggccggggccggcggagcgcccacgcccacccccgggacagctgccccctgggccccggccggtgctgccacctggagaccgtgcaggccaccctggaggacctgggctggagcgactgggtgctgagcccccggcagctgcagctgagcatgtgcgtgggcgagtgcccccacctgtaccggagcgccaacacccacgcccagatcaaggcccggctgcacggcctgcagcccgacaaggtgcccgccccctgctgcgtgcccagcagctacacccccgtggtgctgatgcaccggaccgacagcggcgtgagcctgcagacctacgacgacctggtggcccggggctgccactgcgcctga

SEQ ID NO: 27 - DNA, 924 bp, Artificial Sequence
  Synthetic
atgcccggccaggagctgcggaccgtgaacggcagccagatgctgctggtgctgctggtgctgagctggctgccccacggcggcgccctgagcctggccgaggccagccgggccagcttccccggccccagcgagctgcacagcgaggacagccggttccgggagctgcggaagcggtacgaggacctgctgacccggctgcgggccaaccagagctgggaggacagcaacaccgacctggtgcccgcccccgccgtgcggatcctgacccccgaggtgcggctgggcagcggcggccacctgcacctgcggatcagccgggccgccctgcccgagggcctgcccgaggccagccggctgcaccgggccctgttccggctgagccccaccgccagccggagctgggacgtgacccggcccctgcggcggcagctgagcctggcccggccccaggcccccgccctgcacctgcggctgagccccccccccagccagagcgaccagctgctggccgagagcagcagcgcccggccccagctggagctgcacctgcggccccaggccgcccggggccggcggcgggcccgggcccggaacggcgaccactgccccctgggccccggccggtgctgccggctgcacaccgtgcgggccagcctggaggacctgggctgggccgactgggtgctgagcccccgggaggtgcaggtgaccatgtgcatcggcgcctgccccagccagttccgggccgccaacatgcacgcccagatcaagaccagcctgcaccggctgaagcccgacaccgtgcccgccccctgctgcgtgcccgccagctacaaccccatggtgctgatccagaagaccgacaccggcgtgagcctgcagacctacgacgacctgctggccaaggactgccactgcatc

SEQ ID NO: 28 - DNA, 921 bp, Artificial Sequence
  Synthetic
atgcccggccagggccccgcccccgcccactgcagccccatgctggtgatcctggtgatgctgagctggctgcccagcggcggcgccctgagcctggcccaggagcacctgcccgccttccccggccccagcgacccccacagcagcaccgacgtgagccggatccaggagctgcggaagcggtacgagcacctgcagaccaagctgcggctgaaccagggctgggccgacagcaaccccgacctggtgcccgccacccgggtgcggatcctgacccccaagctgcggctgggcccccggggccacctgcacctgcggatcgcccgggccgacctgaccgccggcctgcccgccgccagccggctgcaccgggccctgctgcggctgagccccaccgagcccagcagctgggacgtgacccggcccctgcagcggcagctgagccgggtgggcagccggacccccaccctgcggctgcggctgctgccccggtgggaccggagccgggccctgcccagcgcccggccccagctggagctgcactggcggccccgggccgcccggggccggcggaacgcccacgcccacgcccgggacggctgccccctgggcgagggccggtgctgccggctgcagagcctgcgggccagcctgcaggacctgggctgggccaactgggtggtggccccccgggagctggacgtgcggatgtgcgtgggcgcctgccccagccagttccggagcgccaacacccacgcccagatgcaggcccggctgcacggcctgaaccccgacgccgcccccgccccctgctgcgtgcccgccagctacgagcccgtggtgctgatgcaccaggacagcgacggccgggtgagcctgacccccttcgacgacctggtggccaaggactgccactgcgtg

SEQ ID NO: 29 - DNA, 927 bp, Artificial Sequence
  Synthetic
atgcccggccccggccccaccccccccatgctgctgatgctgctgatgctgctgatgctgtgctggctgcccagcggcggcgccctgagcctggcccaggagcacctgcccgccttccccggccccagcgaggcccggagcggcaccgacgtgagccggttcgaggagttccggaagctgtacgagcacctgcagacccggctgcggctgaaccagagctgggaggacagcaaccccgaccgggtgatcagcgaggcccaggtgcggatcctgacccccaagctgcggctgggcctgggcggccacctgcacctgcggatcgcccgggccgacctgaccaagggcctgcccgccagcttccggctgcaccgggccctgctgcggctgagccccaccgagctgagcagctgggacgtgacccggcccctgcggcggcagctgagcctgggcggccccggccggcccaccctgcacctgcggctgctgccccagcgggaccggagccccgccgccctgcccagcagcgcccggccccagctggagctgcactggcggccccgggccgcccggggccggcggaacgcccacgcccggagcaaggacgactgccccctgggcgccggccggtgctgccggctgcagagcctgcgggccagcctggaggacctgggctgggccagctgggtggtggccccccgggagctggacgtgcggatgtgcatcggcgcctgccccagccagttccggagcgccaacacccacgcccagatgcaggcccggctgcacggcctgaaccccgacgccacccccgccccctgctgcgtgcccgcccggtacgagcccgtggtgctgatgcaccaggacagcgacggccgggtgagcctgacccccttcgacgacctggtggccaaggactgccactgcctg

SEQ ID NO: 30 - DNA, 900 bp, Artificial Sequence
  Synthetic
atgcggcacttccccatgctgctgctgagctggctgctgctgtggctgccccccggcggcgccctgcccctgacccaggaccacagccccgccttcctgggccccagcggcggccacagcagcctggacgtgagccggttccgggagctgcgggagcggtacgagcacctgcaggcccggctgctgctgaaccagacccaggaggactggaacgccgaccccatccccgtggaccacgtgcggatgctgacccccaagctgcggctgggccccgacggccacctgcggctgcacgccctgcgggccgacctgaccgagggcctgcccgccggcagccggctgcggcaggccctgctgcggctgagcccccaggcccccggcagctgggacctgacccggcagctgcagcggcagctgcggctgggcggccccgccgcccccgccctgagcctgcggctgccccggcggggcggccccagccccgaggccctgcgggccgcccagccccggctggagctgcggtggtggccccccgccgcccggggccggcggggcgcccacgcccacgcccgggacgcctgccccctgggcgagggccggtgctgccggctgctgagcctgcgggccagcatcgaggacctgggctgggccgactgggtggtggccccccgggagctggacgtgcggatgtgcgtgggcgcctgccccggccggttccggagcgccagccggcacgccgaggcccaggcccggctgcacggcctgaagcccgccgccgcccccgccccctgctgcgtgcccgccggctacgcccccgtggtgctgctgcaccgggccgccgacggccgggtggccctgacccccttcgacgacctggtggccgccggctgccactgccag

SEQ ID NO: 31 - protein, 273 aa, Artificial Sequence
  Synthetic
  misc_feature (7)..(7): Xaa can be any naturally occurring amino acid
MQKLQIXVYIYLFMLIVAGPVDLNENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCD

SEQ ID NO: 32 - protein, 307 aa, Artificial Sequence
  Synthetic
  misc_feature (7)..(7): Xaa can be any naturally occurring amino acid
  misc_feature (9)..(11): Xaa can be any naturally occurring amino acid
  misc_feature (19)..(19): Xaa can be any naturally occurring amino acid
  misc_feature (44)..(44): Xaa can be any naturally occurring amino acid
  misc_feature (48)..(49): Xaa can be any naturally occurring amino acid
  misc_feature (81)..(82): Xaa can be any naturally occurring amino acid
  misc_feature (98)..(98): Xaa can be any naturally occurring amino acid
  misc_feature (143)..(143): Xaa can be any naturally occurring amino acid
  misc_feature (157)..(157): Xaa can be any naturally occurring amino acid
  misc_feature (163)..(163): Xaa can be any naturally occurring amino acid
  misc_feature (168)..(169): Xaa can be any naturally occurring amino acid
  misc_feature (200)..(200): Xaa can be any naturally occurring amino acid
  misc_feature (205)..(205): Xaa can be any naturally occurring amino acid
  misc_feature (255)..(255): Xaa can be any naturally occurring amino acid
  misc_feature (263)..(263): Xaa can be any naturally occurring amino acid
  misc_feature (267)..(267): Xaa can be any naturally occurring amino acid
  misc_feature (285)..(287): Xaa can be any naturally occurring amino acid
  misc_feature (307)..(307): Xaa can be any naturally occurring amino acid
MPGQGPXPXXXPMLLLLLXLSWLPSGGALSLAQEHLPAFPGPSXPHSXXDVSRFRELRKRYEHLQTRLRLNQSWEDSNPDXXPAPAVRILTPKLRLGXGGHLHLRIARADLTEGLPAASRLHRALLRLSPTAPSSWDVTRPLXRQLSLGGPRAPALXLRLLPXPDRSXXALPSARPQLELHWRPRAARGRRNAHAHARDXCPLGXGRCCRLQSLRASLEDLGWADWVVAPRELDVRMCVGACPSQFRSANTHAQXQARLHGLXPDAXPAPCCVPASYEPVVLMHXXXDGRVSLTPFDDLVAKDCHCX

SEQ ID NO: 33 - protein, 304 aa, Unknown
  Murine
MAPPALQAQPPGGSQLSFLLFLLLLLLLLSWPSQGDALAMPEQRPSGPESQLNADELRGRFQDLLSRLHANQSREDSNSEPSPDPAVRILSPEVRLGSHGQLLLRVNRASLSQGLPEAYRVHRALLLLTPTARPWDITRPLKRALSLRGPRAPALRLRLTPPPDLAMLPSGGTQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVARGCHCAM